(19) United States
(12) Patent Application Publication
(10) Pub. No.: US 2008/0108930 A1
(43) Pub. Date: May 8, 2008

(54) Methods and Systems for Determining Volume Flow in a Blood or Fluid Conduit, Motion, and Mechanical Properties of Structures Within the Body

(75) Inventors: William F. Weitzel, Ypsilanti, MI (US); Yogesh B. Gianchandani, Ann Arbor, MI (US)

Correspondence Address: BUTZEL LONG, 350 SOUTH MAIN STREET, SUITE 300, ANN ARBOR, MI, 48104, US

(73) Assignee: The Regents of the University of Michigan, Ann Arbor, MI (US)

(21) Appl. No.: 11/931,439

(22) Filed: Oct. 31, 2007

Related U.S. Application Data
(60) Provisional application No. 60/856,589, filed on Nov. 3, 2006.

(51) Int. Cl. A61M 1/34; B01D 61/24

(52) U.S. Cl. 604/5.04; 210/130; 210/646; 210/741; 73/861.42

(57) ABSTRACT

The present invention provides a system for determining blood flow rate in a vessel which communicates blood between two locations of a patient, the system comprising: a conduit in fluid communication with the vessel; at least one sensor in communication with the vessel for determining differential blood pressure (? P) between two or more locations within the vessel; and a processor operably connected to the at least one sensor for processing the ? P to obtain blood flow rate within the vessel.

A method for determining blood flow rate in a vessel which communicates blood between two locations of a patient, the method comprising: diverting blood from the vessel at a diversion point to obtain a flow of diverted blood in a conduit; determining differential blood pressure (? P) of the diverted blood through the conduit; and processing the ? P to obtain blood flow rate within the vessel.

### CROSS-REFERENCE TO PRIORITY APPLICATION

This application claims benefit of U.S. Provisional Application No. 60/856,589, entitled “Methods and Systems for Determining Volume Flow in a Blood or Fluid Conduit, Motion, and Mechanical Properties of Structures Within the Body” and filed Nov. 3, 2006, the content of which is incorporated herein by reference in its entirety.

### STATEMENT REGARDING FEDERALLY SPONSORED RESEARCH OR DEVELOPMENT

The invention was made with Government support under NIH Grant No. 5 K08 DK062848-02. The Government has certain rights to the invention.

### BACKGROUND OF THE INVENTION

This invention relates to the field of hemodynamics, and more particularly to a system and method for measuring blood flow rate in a vessel, such as a hemodialysis access.

Hemodialysis is a process by which blood is passed through an external dialysis circuit to replace the function of a patient's kidney. Blood is removed from the patient's vascular system via an arterial line, is passed through a dialysis filter, and is returned to the patient via a venous line. In order to simplify the withdrawal and return of blood, many dialysis patients have an arteriovenous shunt, or access, surgically created between an artery and vein in a location in the body, such as the upper or lower arm. The access provides a permanent site where the arterial line and venous line can be connected to the patient. A vascular access may be constructed from a native arteriovenous fistula, which is a direct connection of a patient's artery to one of his/her veins, or alternatively may be constructed from a synthetic material, typically polytetrafluoroethylene (PTFE).

While a permanent vascular access provides a convenient connection site for arterial and venous lines, malfunction of such an access is a frequent occurrence in patients receiving chronic hemodialysis. Specifically, unpredictable thrombosis and stenosis in an access causes a reduction in blood flow which necessitates correction through angioplasty or other surgical means. If untreated, low blood flow can cause undesired recirculation in the access, where some part of the freshly dialyzed blood from the venous line flows upstream to the arterial line where it is again filtered. Studies have shown that decreased hemodialysis access flow is associated with an increased risk of access thrombosis and stenosis, such that early detection of an access with a low flow rate is essential in order to prevent more serious complications (see May et al., Kidney Int. 52: 1656-1662, 1997).

Therefore, the importance of sufficient access blood flow has resulted in the emergence of access surveillance as a necessary component in the care of patients on hemodialysis. Surveillance techniques have been developed to detect low blood flow predictive of future thrombosis and stenosis.

An early method of calculating the access flow rate involves occluding the access, placing a needle into the access to monitor the pressure therein, and pumping blood around the occlusion to determine the relationship between blood flow rate and pressure within the access. This intra-access pressure monitoring may be performed either upstream (see Langescheid et al., Dialysis and Transplantation June: 54-55, 1977) or downstream (see Brosman et al., J. Am. Soc. Nephrol. 7: 966-969, 1996) from the occlusion. Unfortunately, occlusion of the access may lead to thrombosis, and placement of the needle or pressure sensor within the access is invasive. Static and dynamic venous pressure monitoring, whereby the pressure within the access is measured with the dialysis blood pump off (static) or on (dynamic), have also been used for surveillance (see Besarab et al., ASAIO J. January-February: 35-37, 1998; Schwab et al., Kidney Int. 36: 707-711, 1989). However, these methods do not correlate well enough with blood flow rate and lack the sensitivity and specificity needed for accurate access surveillance.

At present, the most reliable methods for surveillance of access blood flow utilize conventional Doppler ultrasound (see Stauch et al., Am. J. Kidney Dis. 19: 554-557, 1992; Kirshbaum and Compton, Am. J. Kidney Dis. 25: 22-25, 1995; Findley et al., Radiographics 13: 983-999, 1993; Sands, ASAIO J. January-February: 41-43, 1998; Oates et al., Ultrasound Med. Biol. 16: 571-579, 1990; Sands et al., ASAIO J. 38: M524-M527, 1992) or indicator dilution techniques (see Depner, ASAIO January-February: 38-39, 1998; Krivitski, Kidney Int. 48: 244-250, 1995; Lindsay et al., ASAIO J. January-February: 62-67, 1998).

To evaluate a vascular access using Doppler ultrasound, an ultrasound unit with both imaging and spectral flow Doppler capabilities, termed duplex ultrasonography, is typically utilized. Access blood flow is calculated using the time-velocity integral of a spectrum obtained from a representative area of the access. The cross-sectional area of the access is measured via imaging, and from these measurements volume blood flow is calculated. However, Doppler ultrasound techniques are fraught with sources of operator error, most often associated with the determination of cross-sectional area as well as assumptions about the velocity profile. In addition, conventional Doppler ultrasound is labor intensive and expensive, such that measurements are not usually made with high enough frequency to effectively monitor the onset of reduced access flow. Indicator dilution methods have also been utilized to measure access blood flow. U.S. Pat. No. 5,685,989 issued to Krivitski et al. discloses a dilution technique which uses ultrasonic sensors on the arterial and venous lines. For the measurement of access blood flow, the blood lines are reversed and a temporary recirculation is created. Then, a known quantity of an indicator, such as saline, is injected into the venous line. This dilutes the flow of blood in the access, resulting in Doppler velocity changes measured by the ultrasonic sensor on the arterial line. Because this change is proportional to the concentration of injected saline in the blood, access flow can be calculated. The use of other indicator dilution methods to determine blood flow can be found in U.S. Pat. No. 5,312,550 issued to Hester, U.S. Pat. No. 5,510,716 issued to Buffaloe, IV et al., and U.S. Pat. No. 5,644,240 issued to Brugger. Unfortunately, conditions affecting indicator mixing and recirculation of the indicator through the cardiovascular system can affect the accuracy of results using this method. Furthermore, due to the necessity for the reversal of blood lines during dialysis, dilution techniques are cumbersome and time-consuming.

The present invention exploits the dependence of flow on differential pressure between the dialysis needles when used as a parameter by a processor to determine the flow using knowledge about the geometry and fluid characteristics. The present invention also exploits the decreasing access blood flow within the access between the needles with standard needle placement during dialysis as blood is pumped through the dialysis circuit. The access has a blood flow rate (QA) dependent on numerous factors including systemic blood pressure and central venous pressure (reflecting pressure gradient pre and post access), access geometry (and thereby resistance), and blood viscosity. The access has two needles introduced into its lumen during dialysis; one for the removal of blood (arterial) to pass it through the dialysis circuit and one for the return of blood (venous) to the circulation. The flow through the graft or fistula downstream (QD) from the arterial needle will decrease during dialysis as a function of the blood flowing through the dialysis circuit at a blood pump flow rate (QB). To the extent that the net flow through the system does not change during dialysis, this flow rate through the portion of the access between the dialysis needles during dialysis (QD) will follow the relationship QD=QA−QB.

### SUMMARY OF THE INVENTION

The present invention provides a system for determining blood flow rate in a vessel which communicates blood between two locations of a patient, the system comprising: a conduit in fluid communication with the vessel; at least one sensor in communication with the vessel for determining differential blood pressure (? P) between two or more locations within the vessel; and a processor operably connected to the at least one sensor for processing the ? P to obtain blood flow rate within the vessel.

A method for determining blood flow rate in a vessel which communicates blood between two locations of a patient, the method comprising: diverting blood from the vessel at a diversion point to obtain a flow of diverted blood in a conduit; determining differential blood pressure (? P) of the diverted blood through the conduit; and processing the ? P to obtain blood flow rate within the vessel.

### BRIEF DESCRIPTION OF THE DRAWINGS

Other advantages of the present invention will be readily appreciated as the same becomes better understood by reference to the following detailed description when considered with the accompanying drawings wherein:

FIG. 1a illustrates a hemodialysis system in accordance with the present invention;

FIG. 1b illustrates a further hemodialysis system in accordance with the present invention;

FIG. 2 is an enlarged view of the connections to a hemodialysis access within the system of FIG. 1a;

FIG. 3 is a schematic representation of the hemodialysis system of FIG. 1b;

FIG. 4 is a further schematic of the hemodialysis system of FIG. 1b.

FIG. 5 depicts an alternative schematic representation of the hemodialysis system within the system of FIG. 1;

FIG. 6 schematically illustrates an embodiment of the present invention utilized for single needle dialysis;

FIG. 7 shows an intravascular catheter embodiment of the blood flow rate measuring system of the present invention;

FIG. 8 is a schematic illustration of an electrical equivalent model;

FIG. 9 is a schematic representation of a test circuit and flow phantom;

FIG. 10 shows graphs of modeling functions according to the present invention used to represent the relationship between ? P (pressure) and access flow Q;

FIG. 11 shows a graph of modeling functions according to the present invention used to represent the relationship between ? P (pressure) and access flow Q;

FIG. 12 shows graphs illustrating the summary mean pressure vs. flow relationship;

FIG. 13 are graphs illustrating ? P vs. Re;

FIG. 14 are graphs illustrating mean ? P vs. graft inner diameter at increasing flow rates;

FIG. 15 is a schematic depiction of a patient model;

FIG. 16 is a graph illustrating absolute pressure vs. position within the access;

FIG. 17 are graphs illustrating modeling results determining access flow for 4.76 mm(a) and 6.35 mm(b) diameter access, without geometry or viscosity dependent terms;

FIG. 18 is a graph of modeled flow vs. true flow;

FIG. 19 is a graph of modeled flow vs. true flow; and

FIG. 20 is a graph illustrating differential pressure wave form results of pulsatile flow shifted by turning a pump on.

### DETAILED DESCRIPTION OF THE INVENTION

As required, detailed embodiments of the present invention are disclosed herein; however, it is to be understood that the disclosed embodiments are merely exemplary of the invention that may be embodied in various and alternative forms. The FIGURES are not necessarily to scale, some features may be exaggerated or minimized to show details of particular components. Therefore, specific structural and functional details disclosed herein are not to be interpreted as limiting, but merely as a representative basis for teaching one skilled in the art to variously employ the present invention. U.S. Pat. Nos. 6,167,765; 6,575,927; and 6,709,414 are each incorporated by reference herein.

The present invention provides a system and method for determining the blood flow rate in a vessel, such as a hemodialysis access. Blood flow rate in the vessel is determined by diverting a portion of the blood from the vessel into a conduit, such as an external dialysis circuit, and applying the principle of conservation of mass. The pressure in the vessel is measured at a first point and a second point spatially separated (e.g., downstream of) from the first point. The pressure change between the first point and the second point can then be used to calculate the blood flow rate in the vessel, which represents the net vessel flow rate. Depending on the location and nature of the vessel, net vessel flow rate can indicate such clinically important measures as the functionality of a hemodialysis access, the cardiac output, or the blood being delivered to an extremity.

The present invention includes a method of flow determination using intra-access pressure and its dependence on dialysis pump speed to determine access flow. More particularly, the present invention includes a system and method for determining access flow from intra-access pressure measurements independent of access geometry and blood rheology. The method according to the present invention has the potential to result in an easy to use, operator independent method of access monitoring.

While pressure measurements within the access have been used as an indicator of stenosis (which partially obstructs flow and alters access pressure), none of the currently used methods have used the pressure difference within the blood circuit, particularly within the dialysis graft or fistula, along with knowledge of the blood conduit (access) geometry or other parameters in a mathematical model relating pressure and flow, to estimate flow and used this flow estimation in practice. Decreasing access blood flow rate predicts access stenoses and timely intervention may prevent thrombosis. Flow monitoring also helps stratify thrombosis risk, especially when used in conjunction with other factors or at the appropriate time interval.

Prior systems and methods do not use the pressure difference between arterial and venous needles, measured from the dialysis machine or other device, to determine access flow (velocity or volume flow) when used in conjunction with assumptions about or measurements of the dialysis access geometry (e.g. cross section) and other parameters (e.g. viscosity) or other modeling function based on reference measurements made using other techniques such as ultrasound measurements or otherwise.

The present invention includes, but is not limited to:

1) determining access flow (Q) or flow velocity (v) by making a pressure measurement within the access or other conduit;

2) Using a mathematical relationship between the measured pressure and flow to determine the flow velocity (v) or the volume flow (Q) in the vessel, dialysis access or conduit;

3) Obtaining knowledge about the geometry and fluid characteristics to use in step 2 from some source:
a) measuring velocity within the access or conduit, or
b) measuring flow within the access or conduit, or
c) using the dialysis graft manufacturers measurement of the geometry of the access or conduit, or
d) measuring the geometry of the access or conduit with ultrasound, or other imaging modality, or
e) other methods

4) Obtaining knowledge about the distance between the pressure measurement points from some source;
a) measuring the distance between the dialysis needles, or
b) estimating the distance by inspection, or
c) using historical information about the typical distance, or
d) using historical information about the distance for a given person's access; or
e) others.

According to one aspect of the present invention, the dialysis machine pressure sensors may be used to make pressure measurements to determine the flow. Commonly, pressure readings from dialysis machines report pressure to the nearest 10 mmHg. This is not thought to be of high enough resolution to allow this method to be reduced to practice with sufficient accuracy. Since pressure differences may be on the order of a few mmHg or even less than a mmHg, the sensing mechanisms may need to be modified to allow more precise measurements of pressure to determine the pressure difference between the two locations within the dialysis access (the blood conduit in this case) for useful measurement.

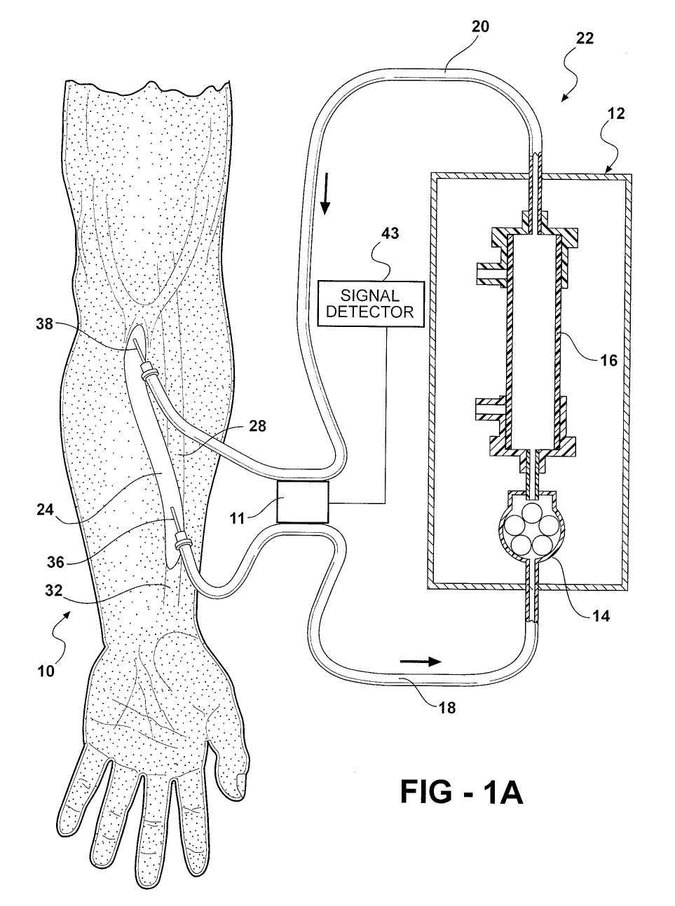

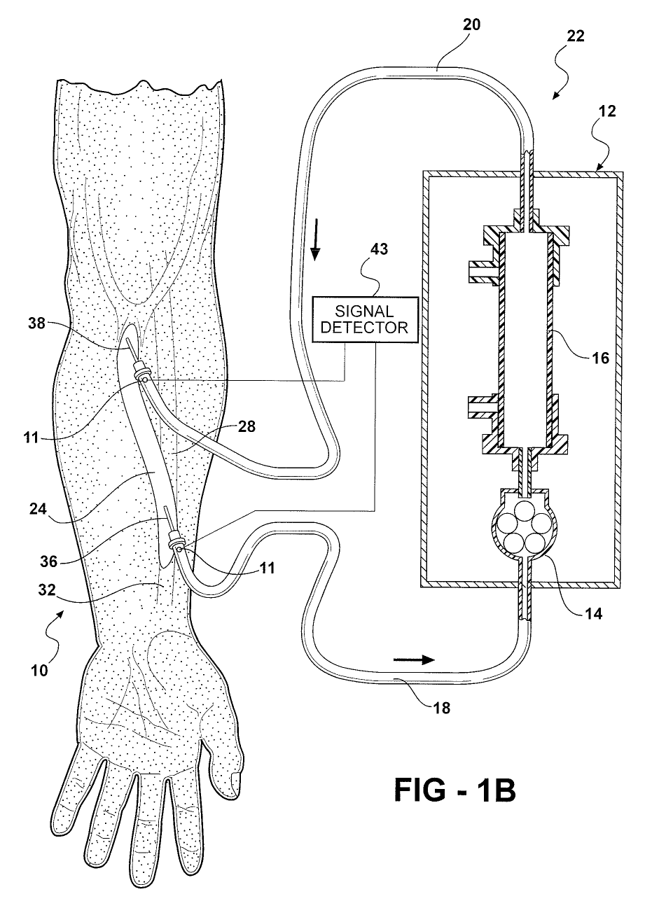

In accordance with the present invention, a hemodialysis system is provided that uses a sensor 11 to detect ? P, where P is pressure and Q is access flow, the hemodialysis system is designated generally by reference numeral 10 in FIG. 1a. Hemodialysis system 10 comprises conventional dialysis equipment 12, including a dialysis pump 14 and a filter 16. The dialysis equipment 12 is provided on one end with an arterial line 18 and on the other end with a venous line 20, each constructed of sterile tubing. The arterial line 18, the dialysis equipment 12, and the venous line 20 form an external dialysis circuit, denoted by reference numeral 22. To perform hemodialysis, dialysis circuit 22 is connected to a patient's vessel, which is depicted in FIG. 1a as an arteriovenous shunt, or access 24. In this embodiment, the arterial line 18 and the venous line 20 are in fluid communication with the pressure sensor 11, which can be a diaphragm which is connected to a signal detector 43.

FIG. 1b provides a further embodiment of the present invention wherein a hemodialysis system is provided that uses two sensors 11 to detect ? P. The hemodialysis system 10 comprises conventional dialysis equipment 12, including a dialysis pump 14 and a filter 16. The dialysis equipment 12 is provided on one end with an arterial line 18 and on the other end with a venous line 20, each constructed of sterile tubing. The arterial line 18, the dialysis equipment 12, and the venous line 20 form an external dialysis circuit, denoted by reference numeral 22. To perform hemodialysis, the dialysis circuit 22 is connected to a patient's vessel, which is depicted in FIG. 1b as an arterial venous shunt, or access 24. In this embodiment, two sensors 11 are disposed within the arterial line 18 and the venous line 20, preferably located at or near the needle hubs. The sensors 11 are also in communication with a signal detector 43 for receiving pressure signals generated by the sensors 11.

The sensor 11 may include one or more sensors to detect the difference in pressure between two points within the conduit. The sensor 11 may be located outside of the body to detect a property within the conduit, for example pressure within the body may be transmitted using a fluidic connection between the intra-luminal location(s) within the conduit to the extra-corporeal sensor to measure intra-luminal pressure (difference). It should be noted that no pump is needed. If a pump is used, resistance in the lines must be known (or assumed) to determine ? P between point 1 and point 2 at the needle tips.

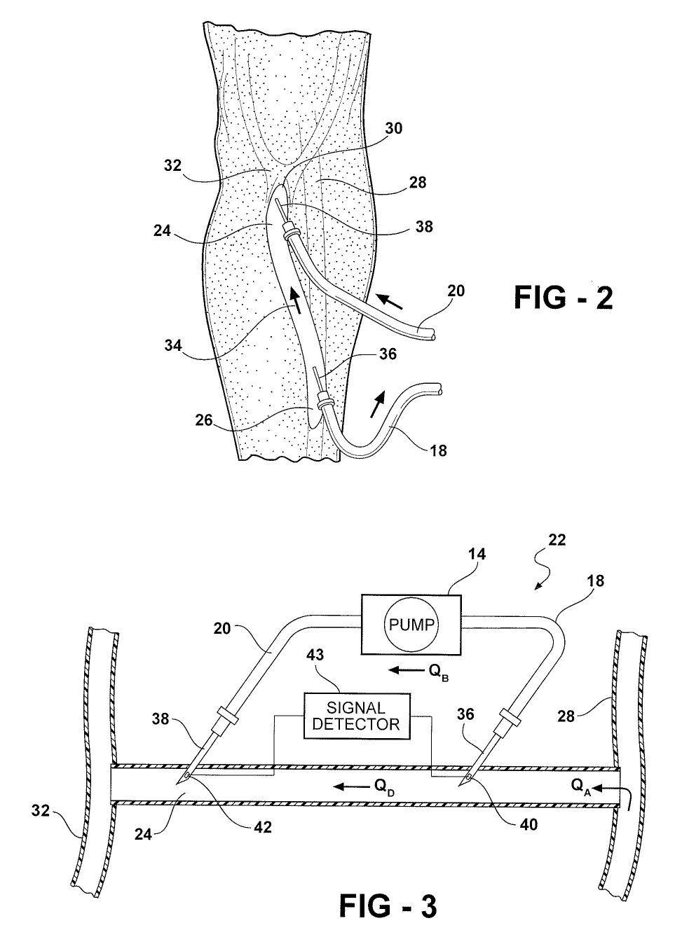

As best shown in FIG. 2, the access 24 has a first end 26 connected to a patient's artery 28 and a second end 30 connected to a patient's vein 32. The access 24 may be an artificial subcutaneous vessel, such as a polytetrafluoroethylene (PTFE) graft, or a native fistula that is surgically created between the artery 28 and the vein 32. The normal direction of blood flow in the access 24 is indicated by arrow 34.

Referring to FIG. 3, the access 24 has two needles introduced into its lumen during dialysis, an arterial needle 36 connected to the arterial line 18 and a venous needle 38 connected to the venous line 20 for the return of blood to access 24. Blood is diverted into dialysis circuit 22 through an arterial needle 36, flows through the arterial line 18 to the venous line 20 while being propelled by pump 14 at a conduit flow rate, and is returned to access 24 via the venous needle 38. A first sensor 40 is provided on or integrated with the arterial needle to generate a signal correlated with the pressure upstream from the venous needle 38 during dialysis. A second sensor 42 is preferably located downstream from the arterial needle 36, on or integrated with the venous needle 38, to generate a signal correlated with the pressure downstream of the arterial needle 36. The sensors 40, 42 are in communication with a signal detector 43 which converts the pressure data from the sensors 40, 42 to calculate access flow rate. The sensors 40, 42 can include ultra-miniature types such as micro-electro-mechanical systems (MEMS), nano-scale, or other small scale sensors known to the person of ordinary skill in the art. The sensors 40, 42 may be in communication with the signal detector 43 via a wireline, wireless, mechanical, electrical, electromagnetic, or other connection. The signal detector 43 may or may not be integrated with the dialysis machine (extracorporeal treatment device).

The needles 36 and 38 are located far enough apart and oriented in such a way that there is sufficient distance between the arterial needle 36 and the venous needle 38 to allow for accurate data collection. Since flow in the vicinity of either the arterial needle 36 or the venous needle 38 will typically be turbulent, sensors 40, 42 are preferably placed at a sufficient distance from each other, on the order of at least 1 cm, to avoid the turbulent flow and obtain a more accurate signal. The needles 36 and 38 are often oriented in the direction of access flow. With this orientation, flow will be moving away from the first sensor 40, allowing signal detection from areas of turbulent flow to be minimized. Such placement of first sensor 40 near or under venous line 20 is facilitated if the first sensor 40 is constructed to be small and have a low profile. In addition, if the first sensor 40 is located in proximity to either arterial 36 or the venous needle 38, then first sensor 40 is preferably directed away from the tips of needles 36, 38, regardless of whether needles 36, 38 are oriented upstream or downstream.

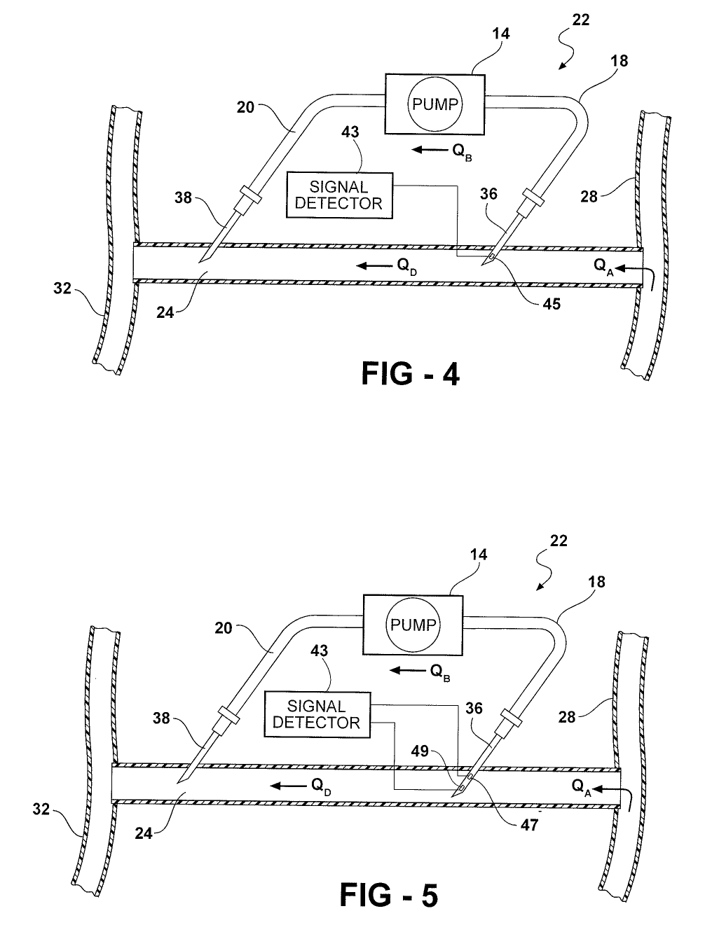

FIG. 4 illustrates a sensor 45 disposed on or integrated with an access needle 36. The sensor 45 may include a combination of sensing elements, such as more than one pressure sensor used to detect ? P which can be related to volume flow or velocity, and may also be ultrasound, Doppler, electromagnetic, Hall effect, chemical sensor, other physical property signal such as viscosity or mass flux sensor, that can be related to flow, velocity, mechanical property or other parameter to be measured according to the present invention which is in communication with signal detector 43.

FIG. 5 illustrates two sensors, associated with the same needle 36, a first sensor 47 and a second sensor 49. The signal detector 43 can detect ? P between points (locations) of sensors 47 and 49.

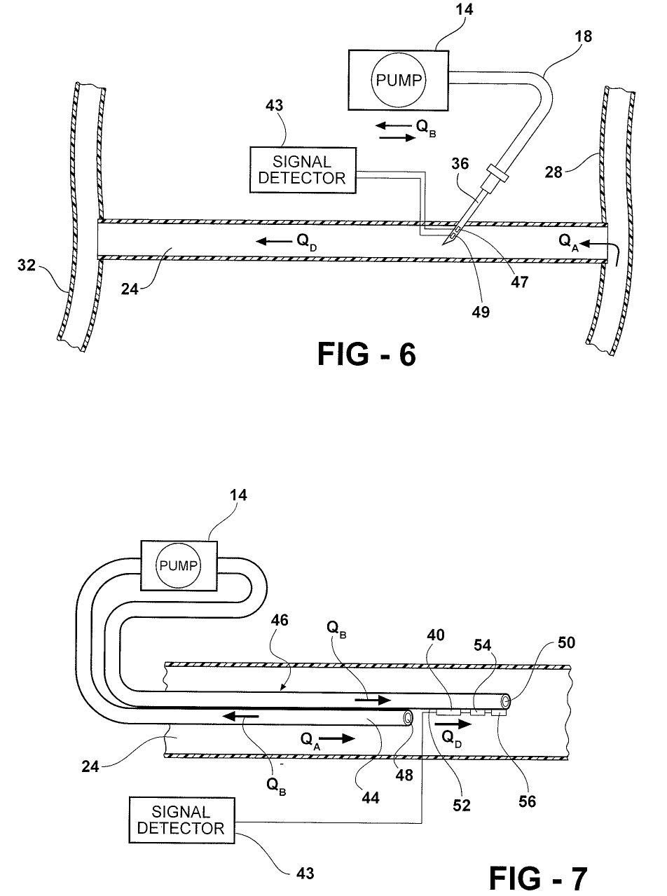

FIG. 6 illustrates an embodiment of the present invention suitable for use in single needle dialysis as described in Van Holder R, Hoenich N, Ringoir S, “Adequacy studies of fistula single-needle dialysis”, Am J Kidney Dis, 10(6); December 1987; 417-426. In this embodiment, two sensors 47, 49 are disposed on or integrated with an access needle 36. The sensors 47, 49 may include a combination of sensing elements, such as more than one pressure sensor used to detect ? P which can be related to volume flow or velocity, and may also be ultrasound, Doppler, electromagnetic, HALL effect, chemical sensor, other physical property signal such as viscosity or mass flux sensor, that can be related to flow, velocity, mechanical property or other parameter to be measured according to the present invention.

In the embodiment shown in FIG. 7, catheter 46 is depicted as a conventional dual lumen catheter having an inlet 48 which allows blood to be diverted from the vessel 24 and into the catheter 46. Blood travels through the catheter 46 at a flow rate Q_{B} generated by an extravascular pump (not shown) similar to the dialysis pump 14, and is returned to the vessel 24 through an outlet 50. However, it should be understood that the return of blood to the vessel 24 via outlet 50 is not required to carry out the method of the present invention. The first sensor 40 is preferably affixed to an outside surface 52 of the catheter 46 downstream from the inlet port 48, more specifically between inlet 48 and outlet 50, to generate the pressure signal. Optionally, sensors 54, 56 may be affixed to outside catheter surface 52 downstream to provide further measures of pressure.

The information from the sensor(s) 40, 42, 45, 47, 49 transmitted to the signal processor 43 which collects and analyses the pressure readings from the needles to calculate the ? P used for flow and other determinations as outlined below. The signal processor 43 can be any suitable electronic device capable of receiving and analyzing the signals transmitted from the sensor(s) 40, 42, 45, 47, 49. The signal processor 43 is preferably extracorporealy disposed.

For embodiments where the pressure sensors 40, 42, 45, 47, 49 are disposed within the vessel 24 or the conduit 18, 20, a miniaturaized pressure sensing device, such as a MEMS, nanoscale, or other small sensor well known in the art can be utilized to minimize or eliminate fluidic resistance. A miniature sensor is defined as a sensor that can be accommodated within the vessel, conduit, or catheter.

It is important to have an observation or measurement of pressure near the vessel. All the capacitance, resistance, and inductance of the pump (and conduit) will affect the measurement. For example, if the measurement is far from the vessel, there will be geometry and time dependent pressure differences from the conduit and pump that will influence the measurement. Therefore, the pressure sensor should ideally be located near to or within the vessel to minimize effects from the conduit and pump.

Since the size of the sensors or sensing mechanisms ideally should not interfere with the flow patterns within the access or vessel or conduit so as not to introduce changes in the differential pressure, the miniature scale sensor enable this measurement method to be realized in practice with the greatest degree of accuracy.

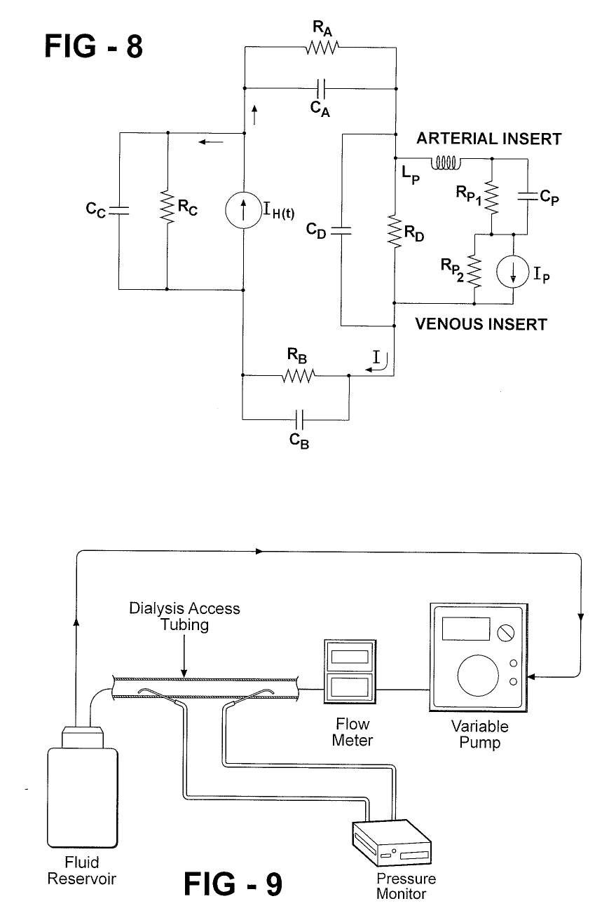

An electrical equivalent model is shown in FIG. 8, which, by way of analogy, can be used to understand the various parameters and factors affecting the hemodialysis system described herein. The model shows a central pump, which is represented by flow I_{H}(ₜ). This could, for example, be used to denote the time-dependent flow through the heart. The flow resistance of the blood vessels upstream and downstream of the dialysis access is denoted by R_{A} and R_{B} respectively. There is an associated capacitance that represents storage, which is necessary for time-dependent analysis. Fluid paths that are in parallel with the dialysis access are denoted by R_{C} and the associated capacitance. In this kind of circuit, inductance represents flow due to momentum effect, and has been left out of the model for simplicity, except for the parasitic cluster of elements near the pump. FIG. 8 shows a Norton equivalent for the pump, though other representations are possible as well.

In FIG. 8, the symbols are defined as:
I_{H}(ₜ)=flow from heart or similar;
R_{A}, C_{A}=impedance upstream of dialysis access;
R_{B}, C_{B}=impedance downstream of dialysis access;
R_{C}, C_{C}=blood flowing in other paths;
R_{D}, C_{D}=impedance of dialysis access;
R_{P}, C_{P}=parasitic resistance and capacitance of pump channels;
I_{P}=flow through dialysis pump;
L_{P}=parasitic inductance;
R_{P1}=parasitic conduit/channel resistance; and
R_{P2}=parasitic pump resistance

In this model, all the “R” terms are linearized equivalent resistances that can be derived from the non-linear flow models at equilibrium/steady state conditions. For time-invariant flows, the capacitance can be ignored. The capacitances denote volume storage in blood vessels and conduits. The elasticity of the blood vessels indicate that both R and C values will depend on local blood pressure (equivalent of voltage in the model above).

The equivalent circuit model illustrates the possibility that pressure/flow measurements of various kinds and at various locations can be made to potentially determine impending access failure or other circulatory problems.

Using the pressure difference between the dialysis needles or along the dialysis access can be used to estimate flow if there is knowledge of the dialysis geometry and factors affecting fluid flow such as blood viscosity. This pressure based flow determination can be used to assist in access monitoring. The pressure drop between needles may be represented by numerous fluid dynamics models representing the blood flow through a dialysis conduit. The pressure in these models depend to varying degrees on polynomial expressions of the flow raised to integer or fractional powers. While many of these take on straightforward algebraic expression, they become rather complicated to implement in clinical practice. In addition to relating flow and pressure, they contain addition terms that include parameters for the dialysis needle separation (or distance along the dialysis access where pressure difference is measured), access diameter (or potentially more complicated forms expressing dialysis access geometry), and factors affecting fluid flow such as blood viscosity. A relationship such as:

? PAV=PV−PA=C1*Q−C2*Q*Q

can describe or model the relationship between pressure and flow. In general, units are not specified for these constants, but one can see that C1 and C2 have different units in this non-linear model. Here, PV is the downstream pressure labeled PV for Venous pressure line on the dialysis machine, PA is the upstream pressure labeled PA for Arterial pressure line, C1 and C2 are constants that depend on access conduit geometry and fluid characteristics, such as viscosity of blood (which may vary with hematicrit and protein content) and Q is the volume flow within the dialysis access (units of volume per unit time).

In using pressure, it is understood that pressure is always a pressure with respect to some reference pressure. Therefore, ? PAV is the pressure difference between the arterial and venous needle site in the dialysis access. Since PV is the relative pressure between the venous needle site and atmospheric pressure, and since PA is the relative pressure between the arterial needle site and atmospheric pressure, PV−PA gives the relative pressure between the two needle sites indirectly using two pressure readings with the same reference pressure (in this case atmospheric pressure), and ? PAV may be determined by direct measurement of the pressure difference between the two points directly using a single pressure measurement transducer.

The geometry of the access, vessel or conduit may be determined by measurement at the time of the pressure measurement or by prior measurements, or by knowledge about the conduit geometry such as in this case of a dialysis graft that has a known inner diameter from manufacturing information. It is understood that limitations in the measurement will depend on the accuracy of the knowledge of the factors that determine C1 and C2 in addition to the accuracy in measurement of the pressures PV and PA. It is also understood that since geometric factors are known, the velocity (v) of blood flow (not just the volume flow (Q) in units of volume per unit time) can be determined since:

Q=v*A

Where A is the cross sectional area of the conduit at a given point or region. This is important since this method can be validated in practice by using other methods such as Doppler ultrasound to determine the velocity of blood flow, then area measurements multiplied by the velocity will give volume flow (the desired access monitoring parameter). Therefore the method allows one to determine velocity using the following model:

PV−PA=C3*v+C4*v*v

where in this case the constants C3 and C4 include the cross-sectional area information.

In general, any mathematical relationship (so called function F) that allows one to map (in a mathematical sense) the two or more pressure measurements to determine the volume flow or velocity in the blood circuit may be used. This may take the general form:

F(PV,PA)=Q

or their inverse relationships. These functions F may be determined from theoretical principles or F (or approximations to F) may be determined from values derived from experiments or clinical data collected and applied to make measurements of Q or v in practice using F or estimation of F.

Other well known relationships relating pressure and flow in a tube are that of Poiseuille's equation:

? P=(128*mu*Q*L)/(pi*D̂4)

where mu is dynamic viscosity, D is graft diameter, L is the length between pressure measurement points (needle separation in the case of dialysis access), and Q is volume flow as above. General fluid dynamics relates pressure to flow in various models. These models typically relate pressure to flow raised to some power or a sum of flow terms to various powers (integer powers or fractional powers or otherwise) in the form of a polynomial, but the mathematical relationship may take any algebraic or numerical or other mathematical form. Using the method according to the present invention, two relationships were selected, one in which pressure is related to the square of flow, and one in which the pressure is related linearly to flow.

A laboratory flow phantom system was assembled to evaluate the method according to the present invention and generate flow and pressure data to test the flow determination algorithms. In these experiments, different access diameters were used (4.35 mm, 6.35 mm, 7.95 mm inner diameter) as well as variations in viscosity (simulating hematocrit 21% and 37%) using ATS fluid and glycerol in water solution. The fluid circuit was assembled to generate measurable flow rates with an adjustable pump (e.g., Masterflex, Vernon Hills, Ill., Console Drive Model 7520-40) with flow rates measured, for example, using a McMillan (Georgetown, Tex.) S-110 digital flowmeter and/or an ATS model (ATS Laboratories, Bridgeport, Conn.) which was calibrated to ensure accuracy with fluids of differing viscosities. Dialysis access diameters were simulated using vinyl tubing. The model flow circuit is depicted in FIG. 9.

The pressure difference between the needles measured at the first sensor and the second sensor will decrease as QB (pump flow) increases and QD decreases. While other observable signals that are predictably related to volume flow may have utility in this method, the present invention focuses on ? P (the pressure difference between the location of the first sensor and the location of the second sensor). The signal ? P can be measured and related mathematically to QB using a modeling function constructed for this signal F(QB) based on the measured values such that ? P=F(QB). This modeling function may take the form of any algebraic or numerical one-to-one function (linear, polynomial, exponential or otherwise), but may not necessarily be one-to-one so long as a suitable inverse can be found in the domain of interest or can be used to estimate or determine a solution. As QD decreases with increasing QB, the signal ? P=F(QB) will decrease. As QD approaches zero, ? P will approach zero, or a known value for _{?} P that corresponds to zero blood flow QD. This is in the idealized case where parasitic resistance and pulsatile flow can be ignored. Other models can be derived that include these factors. For evaluating this method, zero or near zero time-averaged mean ? P will correspond to zero volume flow QD. This value can be defined using the modeling function as the signal S0=F(0). This value for F(0) corresponds to the value for QB=QA since QD=zero. QB at the value QA can be solved by calculating the projected intercept or solution or approximation or estimation of a solution of the modeling function where _{?} P=zero or the known value for ? P corresponding to zero mean flow between the needles. These calculations can be performed numerically by determining the inverse function of the modeling function or by solving them algebraically. To evaluate the method most simply, quadratic and linear form of the relationship between ? P and access flow Q were evaluated, with two dialysis pump speeds (pump on and pump off).

For expression 1:

? P=C*Q, in general,

and:

Poff=C*QA, and

Pon=C*(QA−QB)

for Poff and Pon as the ? P for pump off and pump on, respectively. Solving for the access flow QA gives:

QA=QB/(1−Pon/Poff) (Expression 1)

For expression 2:

? P=C*(QA)̂2

and define:

Poff C*(QA)̂2, and

Pon=C*(QA−QB)̂2

The above also is in the idealized case where parasitic resistance and pulsatile flow can be ignored. Other models can be derived that include these factors. Also, other factors can be introduced into the model that include the effects of the fluidic resistance of the pump and external circuit as well. For Poff and Pon as the ? P for pump off and pump on, respectively. Solving for the access flow QA gives:

QA=QB/(1−v(Pon/Poff)). (Expression 2)

where QA depends on QB and the square root of ratio Pon and Poff. It is to be noted that all of the geometric access and needle position parameters as well as the blood viscosity parameters contained in the term C have been eliminated from expressions 1 and 2 above. While these parameters may be helpful in estimating flow from pressure, the present invention provides a method and derived expression for determining flow from pressure that does not depend on these factors. Again, this is the idealized case where parasitic resistance and pulsatile flow can be ignored.

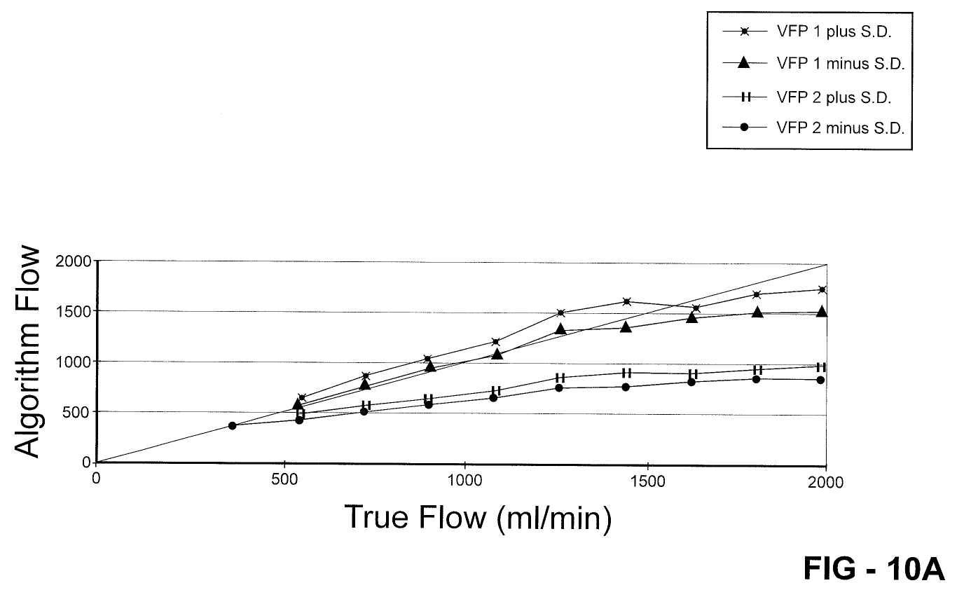

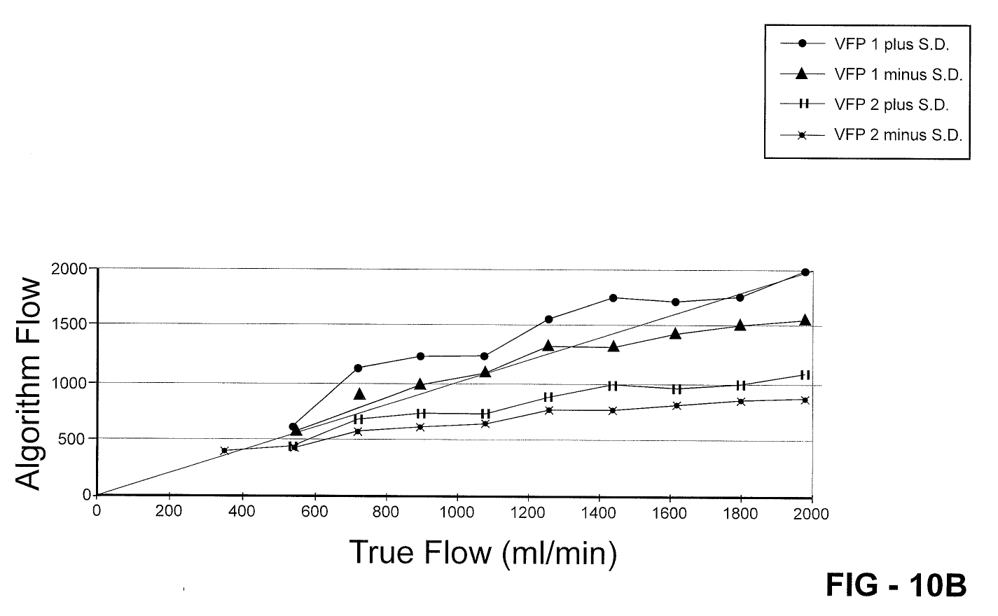

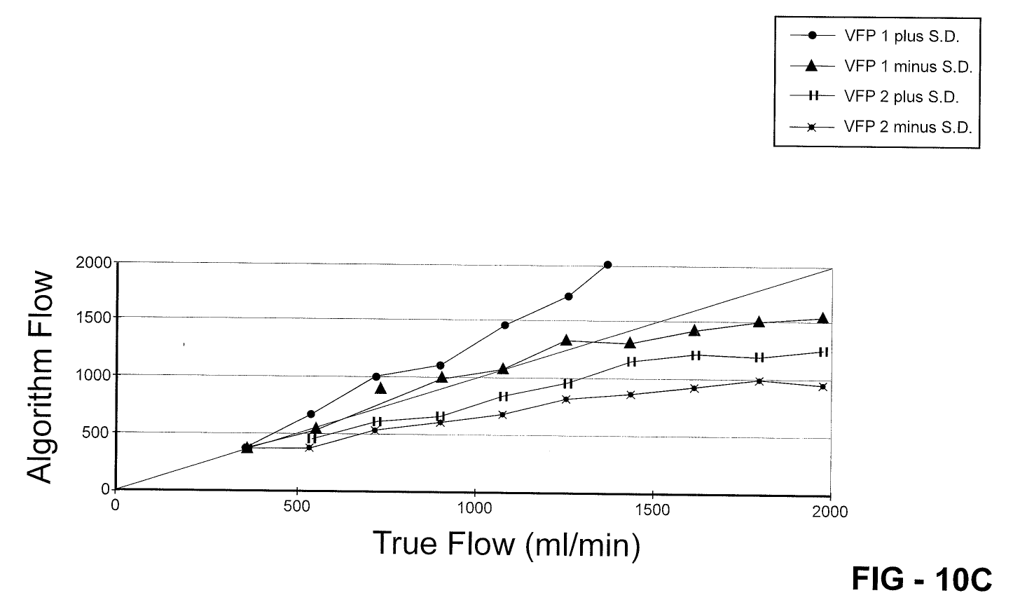

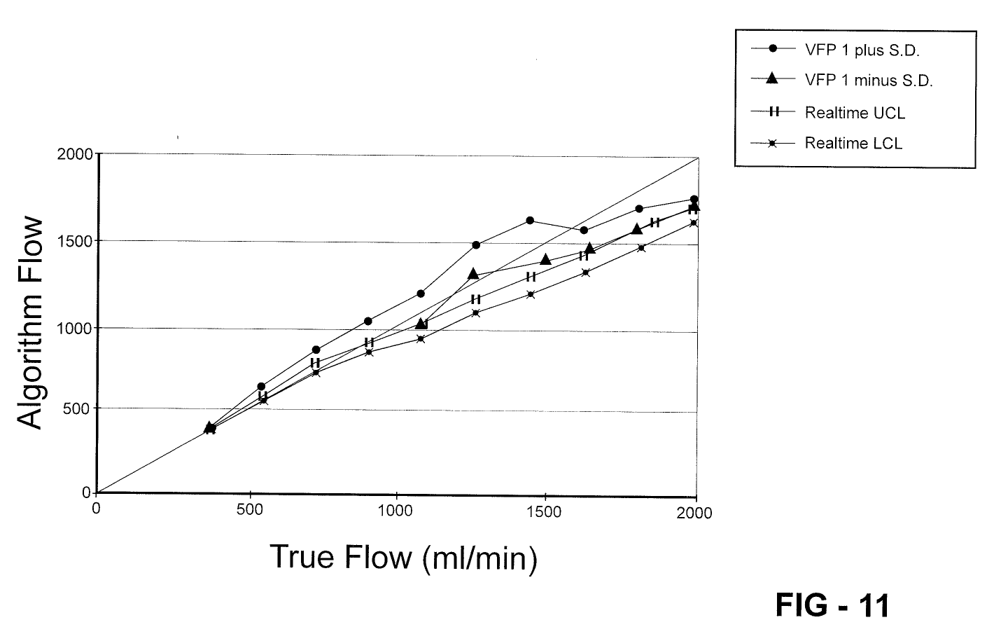

Results are depicted in FIGS. 10 and 11. Note that VFP algorithm 2 is the linear model (expression 1 above), and VFP algorithm 1 is the square model (expression 2 above). FIG. 10 illustrates VFP flow with a 6.35 mm graft for needle separations of 20 cm (FIG. 10a), 10 cm (FIG. 10b), and 5 cm (FIG. 10c). The graph depicted in FIG. 11 shows VFP results for 20 cm needle separation in a 6.35 mm graft with upper and lower confidence limits. It also shows real time estimation of flow using changing Pon with expression 3 along with upper and lower confidence limits. Of note, this real time monitoring value uses the QA values determined with the VFP algorithm as the “base measurement” value for QA obtained rather than the true value of QA where C′ was determined (being 720 ml/min in this case). The reason that the QA used in the real time algorithm was the QA measured by the VFP algorithm was to test the robustness of the method of using variations of Pon in estimating the “true” QA as the method would be used in practice. These results support the validity of this method.

Therefore, the present invention includes a method and apparatus for determining volume flow (Q) or volume flow velocity (v) within a blood circuit within the body by measuring pressure with an extracorporeal pressure measurement device. The method includes obtaining at least one pressure measurement to determine the pressure difference between two or more locations within the blood circuit using the extracorporeal pressure measuring device, and applying a mathematical modeling function that relates the pressure measurement to volume flow (Q) or flow velocity (v). The blood circuit can be a vascular access device for the purpose of receiving extracorporeal treatment, such as a dialysis access device including vascular access devices constructed of prosthetic, autogenous vessels, or other materials.

The pressure measurements can be made using the pressure sensors that are part of a blood treatment device such as a dialysis machine, hemodialysis machine, hemofiltration machine, plasmafiltration device, hemadsorption device, other extracorporeal treatment device or combination of said devices. According to another aspect of the present invention, the pressure measurements can be made using the pressure sensors in a dialysis machine connected to a blood circuit with dialysis needles. The pressure measurements can also be made using the pressure sensors in a dialysis machine that have been modified to measure the pressure difference between the two dialysis lines. The pressure measurement device may measure a derived reading determined by a strain gauge or other mechanical device.

The pressure difference can be determined by direct measurement of the pressure difference between the two locations within the blood circuit. The pressure difference between the two locations in the blood circuit can be determined by measurement using pressure sensors that are externally referenced to a pressure outside the blood circuit such as, but not limited to, atmospheric pressure, then determining the pressure difference between the two or more locations within the blood circuit using the differences between the two or more externally referenced pressure measurements. The pressure difference between the two locations in the blood circuit can be determined substantially simultaneously to accurately approximate a direct pressure difference measurement between the two locations. The pressure difference between the two locations in the blood circuit can be measured as a function of time to determine the variation in flow within the blood circuit that is within the body as a function of time.

There is flow of blood or other fluid flow within the lines where pressure measurements are made that are connected to the blood circuit that is within the body. The resistance in the lines may be known or estimated so that the pressure difference between the two positions in the blood circuit in the body may be determined or estimated to determine the flow.

The pressure difference between the two locations in the blood circuit can be measured to determine flow (Q), and this flow may be used in combination with a pressure referenced externally (PV) to calculate the resistance R in the circuit as PV=Q*R or R=PV/Q. The Q may be replaced by the function dependent on the pressure difference between the two points to determine R, determined from the pressure measurement difference that determines Q. A software or other algorithm can be programmed in the dialysis machine to perform the method according to the present invention to determine flow based on the pressure measurements made in the dialysis machine.

Additional device/methods using sensors on needles with the VF Doppler method in addition to or in conjunction with the above flow determination are contemplated according to the present invention. In addition to determining flow from the ? P, a needle may have 2 or more pressure sensors on one needle to determine the ? P in the blood conduit along the needle. If this is, for example, the downstream needle so that the sensors or sensing mechanism is located between the upstream needle and the outflow hole that is ejecting blood into the dialysis conduit from the downstream needle (the sensors are upstream from the flow of blood back into the conduit), then observing the ? P along the dialysis needle analogous to the methods of VF Doppler, while the dialysis pump speed is varied, allows flow determination with the same error correcting benefits (needle angle, etc) as the VF Doppler method. The flow in the dialysis conduit can be determined using a modeling function analogous to the VF Doppler method. In addition, analysis of the flow waveform (from the ? P) can give diagnostic information the same as Doppler results from a single pump speed and knowledge of or inspection of the pressure (delta pressure or flow) signal over time.

The same can be done using the upstream (arterial) needle if this needle tip faces upstream as it enters the conduit (dialysis access) so that the sensor(s) on the needle section within the blood conduit are located downstream from the diverted channel (arterial blood line in dialysis).

Likewise, the ? P between the two needles may be used (e.g., arterial needle facing upstream and venous needle facing downstream), and sensor for each needle between the location where blood is diverted to the dialysis machine and rejoins the access. Then, the _{?} P at these points will approach zero as the pump speed is increased to approach the access flow, again analogous to the VF Doppler method. A modeling function may be used to determine flow, or the ? P signal may used as a function of time at a single pump speed to access the status of the vascular access.

The method according to the present invention may be advantageous due to its independence of access geometry, needle separation distance, and fluid characteristics such as viscosity (e.g. variable hematocrit, and other factors) which would be required for other pressure based estimations of flow. Also, in contrast to indicator-dilution based methods, no alteration of the patient's blood or dialysis fluid composition are required and no blood line reversal is required for the measurement. In contrast to ultrasound based methods, no imaging is required as in Duplex and transducer placement is needed that might introduce operator dependent factors such as may occur with some other Doppler based measurements. In addition, diagnostic information may be gathered in real time during dialysis including continuous monitoring to detect flow reversal that would lead to recirculation, without altering the treatment at all. And to determine volume flow, only a brief cessation of the pump (a few seconds) would be required in contrast to the several minutes to perform existing measurement methods. In addition, real-time information may be gathered about the pulse waveform that may provide additional information about the access such as flow pattern or indices that may be useful such as augmentation index, other parameters derived from the waveform, or even pulse wave velocity through the access which can yield diagnostic information about the compliance and elastic/mechanical properties of the access.

Real time monitoring of flow may be performed using multiple methods as indicated herein, however, one method that may be practiced would use a relationship that yields flow (QA) as a function of Pon so flow can monitored during dialysis. Because initial experimental data supported the use of expression 2, an expression for real time flow estimation (without altering the pump rate) can yield a parametric value for C′ (geometric and rheologic factors) which can be used for C from the variable flow method.

C′=Poff/QÂ2,

Substituted into Pon=C(QA−QB), and solving for QA gives:

QA=QB+v(Pon/C) (Expression 3)

where QA can be followed in real time without altering pump rate by tracking the square root of the ratio of the ? P with pump on (Pon) and C′ and adding this to the pump rate. An analogous relationship can be determined using expression 1, yielding QA=QB+Pon/C′ should pressure vary linearly with the flow. It should be noted that in practice, during dialysis, it is anticipated that the pump may be briefly paused to recalculate C′ to adjust for factors that may change during dialysis (e.g. ultrafiltration raising the hematocrit and altering viscosity) and then resuming tracking or monitoring QA in real time again. The relationships are not limited to these presented examples of relationships that may be used to allow real time monitoring of QA.

### Experimental

### Experiment I

An vitro study was performed to determine the differential intra-luminal pressure (? P) and flow (Q) relationships in geometry dependent models mimicking arteriovenous graft (AVG) vascular circuits to explore the future development of implantable or extra-corporeal devices using differential pressures to estimate flow for dialysis access monitoring. ? P and Q were obtained using AVG inner diameters of 4.76 mm, 6.35 mm, and 7.95 mm, at separation distances from 2.5 cm to 20 cm, with flows ranging from 0 to 1968 ml/min. Mean and standard deviation values were compared with linear (Poiseuille's), and second order polynomial (Young's) models to model laminar and turbulent flow patterns respectively. Experimental ? P measurement ranged from ±0.015 to 46.95=±0.568 mmHg for the range of studied access diameters at flows from 65 to 1968 mL/min. In conclusion, differential intra-luminal pressure may be useful in flow estimation for dialysis conduits in future implantable or extracorporeal applications. An accuracy of 0.01 mmHg with a range of 0 to 50 mmHg is required for these applications. Further laboratory and clinical studies are planned.

### Methods

A fluid circuit model of the arteriovenous graft (AVG) vascular circuit was developed to study the relationship between the differential pressures between two dialysis access needles (? P) and access flow (Q) (see FIG. 9). The circuit was assembled to generate measurable flow rates, simulating conditions for a vascular access circuit. A Masterflex Console Drive non-pulsatile blood roller pump (Cole Parmer, Vernon Hills, Ill.) was utilized to draw a glycerol-based fluid, with a kinematic velocity of 0.029 cm²/s (corresponding to a hematocrit of approximately 37%), from a fluid reservoir. The fluid was channeled to a Gilmont flow meter (Thermo Fisher Scientific), which was calibrated using the 37% glycerol solution. The scale division of the flow meter is 1 mm, with a range of 0-100 mm. The accuracy of the flow meter is ±5% of the reading or 2 mm of scale length, whichever is greater. The repeatability of the flow meter is ±0.5 scale division, whichever is greater. The fluid subsequently flowed through polyvinyl tubing back to the fluid reservoir before returning to the pump in a closed circuit (FIG. 9). Commonly used AVG inner diameters range from 5-7 mm; therefore, 4.76 mm ( 3/16″), 6.35 mm (¼″), and 7.95 mm ( 5/16″) polyvinyl tubing was utilized for the experiments.

To measure ? P, two 16-gauge needles were placed at needle separation distances of 2.5 cm, 5 cm, 10 cm, 15 cm, and 20 cm from one another within the circuit. The needles were primed with the 37% glycerol solution, and a digital pressure monitor (Validyne model PS409, Northridge, Calif.) was used to directly measure ? P between the “upstream” and “downstream” needles, in mmHg. During steady-state flow, the pressure monitor was observed for 20-30 seconds until the reading stabilized and the pressure reading was recorded. At each flow and needle separation the mean of ten separate measurements was used for data analysis.

In actuality, this flow system is very complicated with the pump, vessels, and conduits all having associated resistance, capacitance, and inductance. Idealized models are considered herein; more complex models can be derived and utilized that include additional factors. Two well-established steady flow models were utilized to determine which most closely approximates the relationship between ? P and Q. Steady flow models are first-order approximations and are used in this first set of experiments to determine the experimental values and compare with theoretical ? P−Q relationship prior to future pulsatile flow studies. The Navier-Stokes differential equations provide a complete mathematical description of incompressible Newtonian fluids. One of the best described solutions for laminar flow through a straight circular tube of constant cross section is the Hagen-Poiseuille (hereafter, Poiseuille) equation. This equation for laminar flow was evaluated, as follows:

$$\text{?} P = \frac{128 μ Q {L}_{G}}{\text{?} {D}_{G}^{4}} \qquad ( eq . 1 )$$

in which μ is the dynamic viscosity of the liquid, L_{G} is the length of the graft, and D_{G}⁴ refers to the inner diameter of the graft raised to the 4th power. With this equation, the relationship between ? P and Q is linear. For each inner tube diameter and at each distance of separation, ten measurements were taken at each flow rate. The mean, standard deviation, and con-elation coefficient values between Poiseuille's model and the experimental data were calculated.

Similarly, Young's general expression for a flow rate-dependent pressure drop between two separate locations when a liquid flows through a channel was evaluated:

? P=RₐV+R_{b}V² (eq. 2)

where ? P represents the pressure difference between the downstream and upstream locations respectively, V is area-averaged flow velocity in an unobstructed vessel, and Rₐ and R_{b} are coefficients that depend on obstacle geometry and fluid properties. Young's expression was chosen as one of the simplest models incorporating higher order terms (Q raised to the second power) that may be used to characterize turbulent flow that may result from higher velocity flow conditions with higher Reynolds numbers, geometry induced flow disturbances from vessel diameter change or intraluminal irregularities, as well as cannulas within the flow path.

As the diameter of the graft tubing for each separate experiment remained constant, the relationship between V and Q can be represented as follows:

Q=V(?D²/4) (eq. 3)

Substituting (eq. 3) into (eq. 2) yields the following:

^{?}P=C₁Q+C₂Q² (eq. 4)

where C₁=R_{b}/(? D²/4) and C₂=Rₐ/(? D²/4).

Subsequently, to evaluate whether the data are best represented by a linear or second order polynomial equation, the data were fitted to Poiseuille's and Young's equations, respectively. Correlation coefficients were calculated to evaluate the fit of the data to each model.

To establish dynamic similitude between our in vitro model and the in vivo AVG circuit, Reynold's numbers were calculated for each flow rate and for each of the three separate AVG inner diameters based on the expression.

$$Re = \frac{ρ v L}{μ} \qquad ( eq . 5 )$$

where ρ is the density of the fluid, v is the velocity, L is the characteristic length and μ is the dynamic viscosity. Based upon a personal communication with Steven A Jones, PhD, (Li, T, Gianchandi R Y, Gianchandi Y B, “Micromachined bulk PZT tissue contrast sensor for fine needle aspiration biopsy”, Lab Chip 7; 2007; 179-185) Density ρ=1090.04 kg/m³ and viscosity=μ=0.0032 kg/m·s. The characteristic length L is the inner diameter, D, of the tube: 4.76 mm; 6.35 mm, 7.95 mm, and velocity v=Q/S=4Q/πD².

Substituting these parameters into equation (5) with varied v, D and μ, allowed the Reynolds condition of the fluid in this experiment to be calculated.

### Results

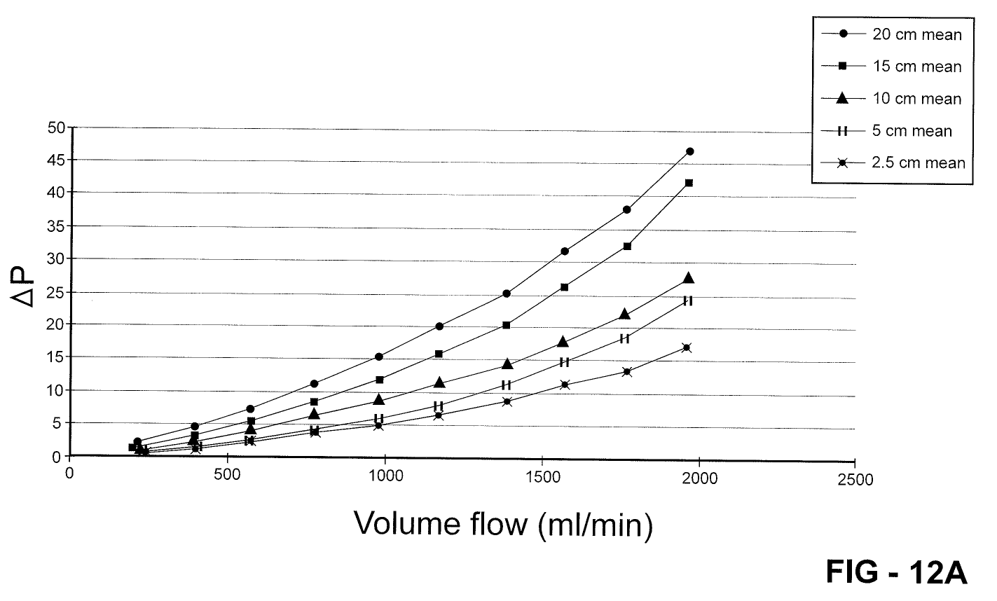

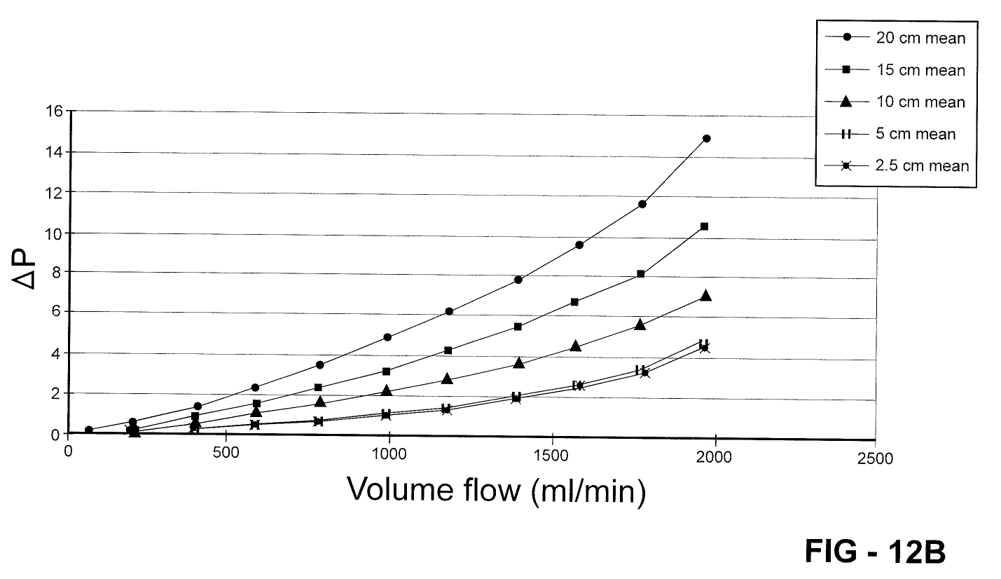

For the 4.76 mm (FIG. 12a) and 6.35 mm (FIG. 12b) inner diameter polyvinyl tubes, the data representing mean ? P values (±SD) at volume flow rates from 65 ml/min to 1968 ml/min at needle separations of 2.5 cm, 5 cm, 10 cm, 15 cm, and 20 cm are shown in Table I and FIG. 12.

TABLE I
Summary Data of Mean ? P (+/− S.D.) vs. Q

| Inner | N.S. | Mean Volume Flow Rate (mL/min, calibrated for 37% glycerol solution) | | | | | |
|---|---|---|---|---|---|---|---|
| Diam | (cm) | 65 | 197.5 | 305 | 575 | 775 | 980 |
| 4.76 mm | 20 | 0.505 +/− 0.049 | 1.825 +/− 0.067 | 4.33 +/− 0.0823 | 7.445 +/− 0.142 | 11.17 +/− 0.163 | 15.28 +/− 0.187 |
| | 15 | 0.415 +/− 0.58 | 1.425 +/− 0.088 | 3.2 +/− 0.131 | 5.41 +/− 0.147 | 8.34 +/− 0.161 | 11.745 +/− 0.116 |
| | 10 | 0.23 +/− 0.054 | 0.855 +/− 0.059 | 2.23 +/− 0.1 | 4.22 +/− 0.137 | 6.385 +/− 0.21 | 8.755 +/− 0.44 |
| | 5 | 0.185 +/− 0.024 | 0.68 +/− 0.089 | 1.685 +/− 0.094 | 2.73 +/− 0.048 | 4.11 +/− 0.19 | 5.92 +/− 0.204 |
| | 2.5 | 0.155 +/− 0.36 | 0.565 +/− 0.078 | 1.405 +/− 0.072 | 2.505 +/− 0.069 | 3.89 +/− 0.129 | 4.91 +/− 0.129 |
| 6.35 mm | 20 | 0.175 +/− 0.048 | 0.665 +/− 0.115 | 1.46 +/− 0.117 | 2.43 +/− 0.116 | 3.58 +/− 0.103 | 4.86 +/− 0.143 |
| | 15 | 0.175 +/− 0.063 | 0.53 +/− 0.134 | 0.925 +/− 0.079 | 1.57 +/− 0.067 | 2.31 +/− 0.088 | 3.22 +/− 0.079 |
| | 10 | 0.045 +/− 0.06 | 0.205 +/− 0.37 | 0.62 +/− 0.042 | 1.125 +/− 0.035 | 1.64 +/− 0.039 | 2.215 +/− 0.088 |
| | 5 | 0 | 0.11 +/− 0.031 | 0.355 +/− 0.055 | 0.55 +/− 0.126 | 0.81 +/− 0.15 | 1.18 +/− 0.14 |
| | 2.5 | 0.06 +/− 0.052 | 0.145 +/− 0.03 | 0.315 +/− 0.024 | 0.555 +/− 0.049 | 0.82 +/− 0.042 | 1.105 +/− 0.055 |
| 7.95 mm | 20 | 0.07 +/− 0.059 | 0.185 +/− 0.034 | 0.435 +/− 0.063 | 0.76 +/− 0.032 | 1.11 +/− 0.041 | 1.505 +/− 0.037 |
| | 15 | 0.04 +/− 0.021 | 0.115 +/− 0.024 | 0.295 +/− 0.037 | 0.635 +/− 0.06 | 0.99 +/− 0.74 | 1.43 +/− 0.67 |
| | 10 | 0.035 +/− 0.024 | 0.105 +/− 0.028 | 0.17 +/− 0.042 | 0.245 +/− 0.043 | 0.4 +/− 0.058 | 0.72 +/− 0.092 |
| | 5 | 0.015 +/− 0.015 | 0.115 +/− 0.24 | 0.215 +/− 0.024 | 0.315 +/− 0.034 | 0.435 +/− 0.047 | 0.595 +/− 0.015 |
| | 2.5 | 0 | 0.05 +/− 0.073 | 1.145 +/− 0.028 | 0.275 +/− 0.0264 | 0.395 +/− 0.028 | 0.59 +/− 0.021 |

| | | Mean Volume Flow Rate (mL/min, calibrated for 37% glycerol solution) | | | | |
|---|---|---|---|---|---|---|
| Inner Diam | N.S. (cm) | 1173.333333 | 1386.666667 | 1573.095238 | 1770.565476 | 1968.035714 |
| 4.76 mm | 20 | 19.88 +/− 0.266 | 25.2 +/− 0.437 | 31.62 +/− 0.561 | 38.05 +/− 0.618 | 46.95 +/− 0.568 |
| | 15 | 15.88 +/− 0.312 | 20.58 +/− 0.513 | 26.17 +/− 0.211 | 32.26 +/− 0.533 | 42.14 +/− 1.036 |
| | 10 | 11.495 +/− 0.844 | 14.22 +/− 0.193 | 17.8 +/− 0.262 | 21.99 +/− 0.363 | 27.65 +/− 0.67 |
| | 5 | 7.9 +/− 0.156 | 11.14 +/− 0.383 | 14.94 +/− 0.425 | 18.56 +/− 0.851 | 24.07 +/− 0.8 |
| | 2.5 | 6.5 +/− 0.133 | 8.72 +/− 0.114 | 11.19 +/− 0.251 | 13.49 +/− 0.29 | 17.08 +/− 0.402 |
| 6.35 mm | 20 | 6.26 +/− 0.158 | 7.81 +/− 0.256 | 9.65 +/− 0.227 | 11.68 +/− 0.312 | 15.04 +/− 0.455 |
| | 15 | 4.31 +/− 0.099 | 5.47 +/− 0.1418 | 6.72 +/− 0.155 | 8.24 +/− 0.157 | 10.59 +/− 0.26 |
| | 10 | 2.86 +/− 0.143 | 3.66 +/− 0.177 | 4.56 +/− 0.084 | 5.56 +/− 0.09 | 7.03 +/− 0.21 |
| | 5 | 1.49 +/− 0.088 | 2 +/− 0.141 | 2.55 +/− 0.268 | 3.23 +/− 0.40 | 4.47 +/− 0.488 |
| | 2.5 | 1.425 +/− 0.132 | 2.01 +/− 0.15 | 2.69 +/− 0.201 | 3.405 +/− 0.183 | 4.81 +/− 0.246 |
| 7.95 mm | 20 | 1.955 +/− 0.055 | 2.46 +/− 0.045 | 3.15 +/− 0.071 | 4.025 +/− 0.042 | 4.835 +/− 0.047 |
| | 15 | 1.76 +/− 0.96 | 1.98 +/− 0.92 | 2.5 +/− 0.047 | 2.94 +/− 0.07 | 3.76 +/− 0.097 |
| | 10 | 1.195 +/− 0.059 | 1.63 +/− 0.082 | 1.84 +/− 0.06 | 2.22 +/− 0.091 | 2.77 +/− 0.067 |
| | 5 | 0.85 +/− 0.075 | 1.21 +/− 0.056 | 1.57 +/− 0.053 | 1.7 +/− 0.047 | 1.82 +/− 0.025 |
| | 2.5 | 0.725 +/− 0.035 | 0.925 +/− 0.059 | 1.2 +/− 0.04 | 1.455 +/− 0.044 | 1.595 +/− 0.109 |

For each of the three tubes of varying inner diameter, ? P increased as the volume flow rate increased. For example, ? P increased from 8 mmHg at a flow of ˜600 ml/min to >45 mmHg at 1968 ml/min at a needle separation of 20 cm. At a needle separation of 2.5 cm, ? P was 3 mmHg at a flow of ˜600 ml/min, demonstrating that as the distance between the two pressure-sensing needles increased, there was a consistent increase in the measured pressure difference. The curves were noted to be non-linear, with an apparent polynomial ? P dependence on flow rate. This relationship appeared to be more pronounced at needle separations greater than 2.5 cm.

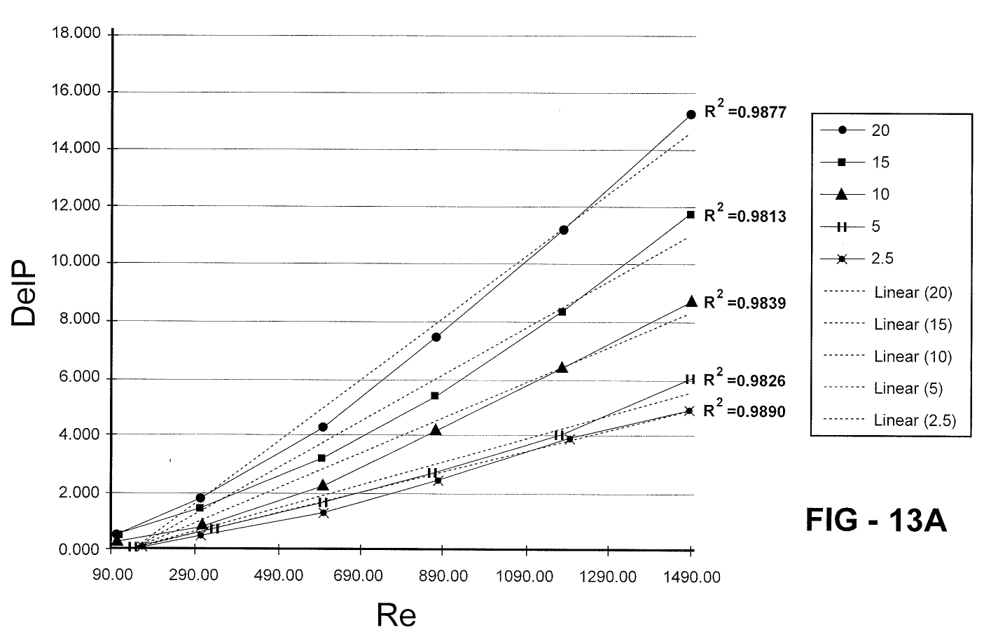

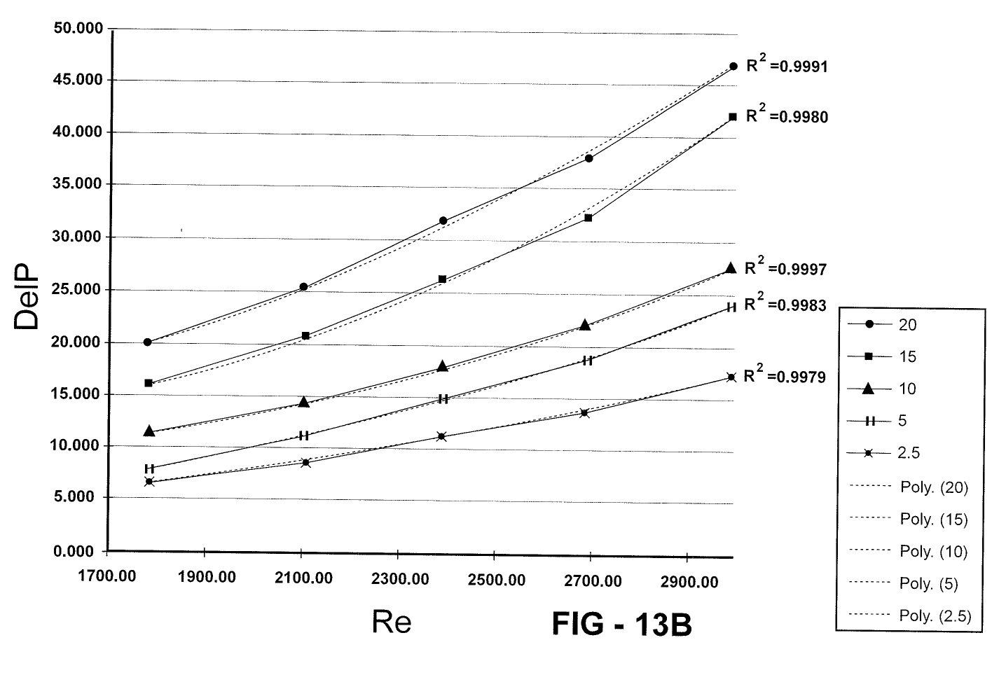

The transition between laminar and turbulent flow usually occurs in flow conditions where the Reynold's number is approximately 1500. To further quantify the ? P−Q relationships, the data for each of the three different diameter tubes were matched to Poiseuille's (laminar flow) (see FIG. 13a) and Young's (turbulent flow) Isee FIG. 13b) equations for Reynolds numbers less than and greater than an approximate transitional value of 1500 respectively. Representative graphs for the 4.36 mm inner diameter data are shown in FIGS. 13a and 13b.

The data comparing the correlation coefficients for Poiseuille's and Young's equations vs. the experimental data for the 7.95 mm, 6.35 mm and 4.76 mm inner diameter tubes are shown in Table II.

TABLE II
R² comparison between Poiseuille's and Young's equations

| | 7.95 mm | | 6.35 mm | | 4.76 mm | |
|---|---|---|---|---|---|---|
| Separation cm) | Poiseuille's | Young's | Poiseuille's | Young's | Poiseuille's | Young's |
| 20 | 0.9216 | 0.9975 | 0.9324 | 0.9961 | 0.9351 | 0.9991 |
| 15 | 0.9484 | 0.9928 | 0.9211 | 0.9968 | 0.8992 | 0.9966 |
| 10 | 0.8918 | 0.9921 | 0.9275 | 0.9975 | 0.9257 | 0.9976 |
| 5 | 0.9357 | 0.9828 | 0.8449 | 0.9855 | 0.8697 | 0.9962 |
| 2.5 | 0.9443 | 0.9962 | 0.8733 | 0.9891 | 0.9138 | 0.9974 |

Table III displays the calculated Reynolds numbers for our experiment. For the 4.76 mm tube, Reynold's numbers were less than 2100 for all flows <1387 mL/min, and for the 6.35 mm inner diameter, only the 1968 mL/min flow demonstrated a Reynolds number >2100. All Reynolds numbers were <2100 for the 7.95 mm inner diameter tube.

| | Flow rate (ml/min) | | | | | | | | | | |
|---|---|---|---|---|---|---|---|---|---|---|---|
| | 65 | 198 | 395 | 575 | 775 | 980 | 1173 | 1387 | 1573 | 1771 | 1968 |
| 4.76 mm | 100 | 303 | 605 | 881 | 1188 | 1502 | 1798 | 2125 | 2411 | 2714 | 3017 |
| 6.35 mm | 75 | 227 | 454 | 661 | 890 | 1126 | 1348 | 1593 | 1807 | 2034 | 2261 |
| 7.95 mm | 60 | 181 | 363 | 528 | 711 | 899 | 1077 | 1273 | 1444 | 1625 | 1806 |

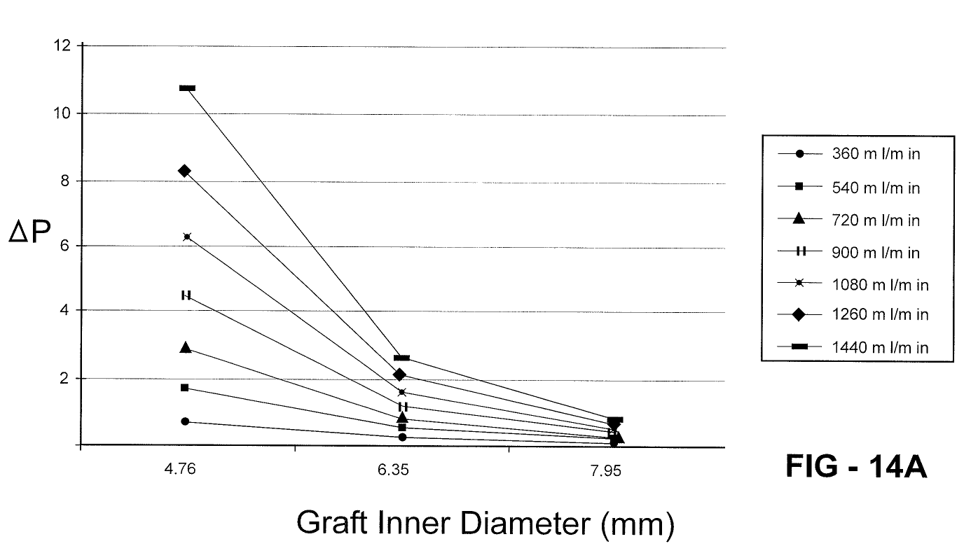

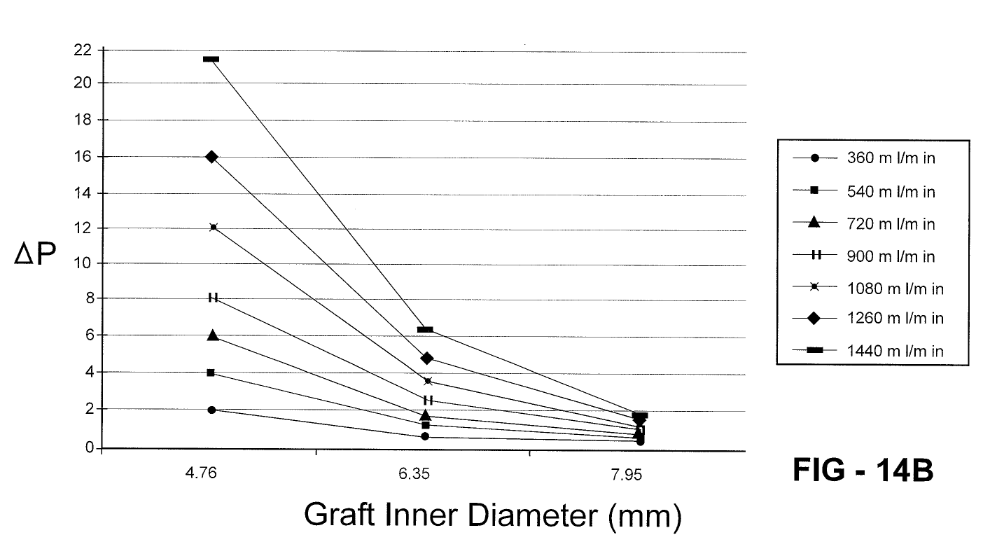

As is seen in FIG. 14a and 14b, ? P increases with the distance between the two access needles. This relationship becomes more pronounced as the access flow increases, with the magnitude of the mean ? P values being substantially greater utilizing the 4.76 mm vs. the 7.95 mm inner diameter tubes (FIGS. 14a and 14b).

Data is provided investigating the possibility of using differential pressure monitoring to estimate access flow for dialysis access monitoring, with the goal of utilizing Micro-Electro-Mechanical Systems (MEMS) pressure sensors integrated within the shaft of dialysis needles. Data derived experimentally evaluating pressure-flow relationships with computational fluid dynamics (CFD) were used to devise and test a method of estimating access flow using differential pressure and variation with dialysis pump speeds (variable flow) that diminishes dependence on geometric factors and fluid characteristics. CFD modeling suggested turbulent needle effects were greatest within 1 cm of the needle tips. Utilizing linear, quadratic and combined variable flow algorithms, dialysis access flow was estimated using geometry independent models and an experimental dialysis system with the pressure sensors separated from the dialysis needle tip by distances ranging from 1 to 5 cm. Real time differential pressure wave form data were also observed during the mock dialysis treatment, which may be useful in detecting low or reversed flow within the access.

### Experiment II

### Materials and Methods

### Experimental System

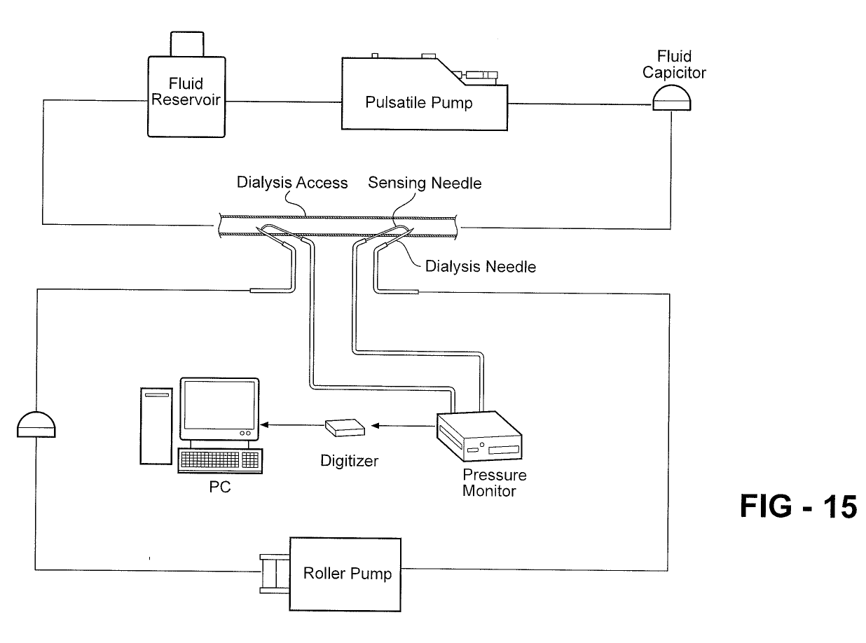

A laboratory flow phantom system was constructed using two parallel fluid conduits to simulate the patient blood circuit communicating in parallel with the dialysis blood pump circuit to test the geometry independent algorithms for flow determination. In these experiments, different access diameters were used (4.76 mm and 6.35 mm inner diameter) to approximate arteriovenous graft inner diameters, as well as a glycerol-containing solution to simulate the viscosity of blood at 37% hematocrit. The dialysis circuit was assembled to generate measurable flow rates with an adjustable non-pulsatile roller pump (Masterflex Cole Parmer, Vernon Hills, Ill. Console Drive Model 7520-40) with flow rates measured using a McMillan (Georgetown, Tex.) S-110 digital flowmeter and a Gilmont flow meter (Thermo Fisher Scientific) which was calibrated in our laboratory to ensure the accuracy of simulated dialysis pump speeds ranging from zero to 500 ml/min. The dialysis circuit was connected to the dialysis graft with 15 gauge dialysis needles (Sysloc, JMS Singapore PTE LTD, Singapore). The dialysis access was simulated using vinyl tubing (Watts Water Technologies, North Andover, Mass.)). The patient blood circuit was modeled using a Harvard Apparatus pulsatile adjustable blood pump (Holliston, Mass.) in series with a bubble trap from ATS Laboratories (Bridgeport, Conn.) to act as a large capacitance vessel. This was in series with the access graft which had been cannulated with the dialysis needles from the dialysis circuit. A downstream air trap was also located within the patient circuit. Pressure sensing within the conduit was achieved using 21 gauge spinal needles positioned with needle tips 5 cm, 2 cm and 1 cm from the upstream facing arterial needle and the downstream facing venous needle tip and pressures determined using a digital pressure monitor (Validyne model PS409, Northridge, Calif.) with digital data download to a PC using data acquisition hardware and software (DATAQ Instruments, Inc, Akron, Ohio). The model flow circuit is depicted in FIG. 15.

Experimental data were collected at varying pulsatile pump speeds of 400, 800, and 1200 ml/min simulating these dialysis access flow rates and the dialysis pump speeds were varied from 0 to 400 ml/min simulating dialysis pump off and on conditions respectively for each access diameter (4.76 and 6.35 mm), with 20 cm dialysis needle separation, at variable pressure sensor needle distances from the intraluminal dialysis needle tip ranging from 1 to 5 cm. Fluid viscosity was 0.29 centistokes corresponding to hematocrit of 37%.

### Derivation of Geometry Independent Models:

The pressure drop between needles can be represented by numerous fluid dynamics models representing the blood flow through a dialysis conduit. The pressure in these models depends to varying degrees on polynomial expressions of the flow raised to integer or fractional powers. While many of these take on straight forward algebraic expressions, they become rather complicated to implement in clinical practice. The reasons leading to difficult implementation are that, in addition to relating flow and pressure, they contain additional parameters such as the dialysis needle separation (or distance along the dialysis access where pressure difference is measured), access diameter (or potentially more complicated forms expressing dialysis access geometry), and factors affecting fluid flow such as blood viscosity. With any of these relationships, it is understood that pressure is always a pressure with respect to a reference pressure. Therefore, if needle pressure is used, the differential pressure between sensors (? PAV) is the pressure difference between the arterial (PA) and venous (PV) needle site in the dialysis access. Since PV, as it is used in dialysis access monitoring currently, is the relative pressure between the venous needle site and atmospheric pressure and since PA is the relative pressure between the arterial needle site and atmospheric pressure, PV−PA gives the relative pressure between the two needle sites indirectly using two pressure readings with the same reference pressure (in this case atmospheric pressure) and ? PAV may be determined by direct measurement of the pressure difference between the two points directly using a single pressure measurement transducer.

In general, any mathematical relationship (so called function F) that allows one to map (in a mathematical sense) the two or more pressure measurements to determine the volume flow (Q) or velocity (v) in the blood circuit may be used. This may take the general form:

F(PV,PA)=Q (equation 1)

Alternatively, their inverse relationships may be utilized. These functions may be determined from theoretical principles, or F (or approximations of F) may be determined from values derived from experiments or collected clinical data and applied to make measurements of Q or v in practice using F or an estimation of F.

Using the previously mentioned well known equation relating pressure and flow in a tube is that of Poiseuille's equation:

? P=(128*μ*Q*L)/(?*D̂4) (equation 2)

where μ is dynamic viscosity, D is graft diameter, L is the length between pressure measurement points (needle separation in the case of dialysis access), and Q is volume flow as above. A pulsatile-flow model relating pressure to flow is not used here; rather, we employ a first-order approximation with steady flow was employed to test the method of measurement being evaluated. Based on theoretical grounds of using laminar flow with linear pressure-flow relationships and our experimental system showing pressure-flow relationships fitting a second order polynomial, two relationships to test were selected, one in which pressure is related to the square of flow and one in which the pressure is related linearly to flow. Other mathematical relationships may take alternative algebraic, numerical, or other mathematical forms.

### Using Diverted Dialysis Pump Flow to Determine Access Flow.

Methods that exploit the decreasing access blood flow between the needles within the access during dialysis as blood is pumped through the dialysis circuit take advantage of changes in pressure within this segment of the access. The effects of needle tip flow must be considered whenever the needle tip flow disturbance is near the pressure transducer; precisely how near or far the transducer must be from the needle tip must be determined from modeling, such as is computational fluid dynamics and experimental results, such as those presented in this experiment.

One physical system exploiting this method involves pressure transducers integrated on the outside of the shaft and the measurement method outlined below will be tested with needle designs in the future based on the experimental results presented in this study. A MEMS manufacturing method referred to as micro Electro-Discharge Machining (EDM) has been used for three dimensional machining of needles which can cut cavities in needle shafts for MEMS sensor integration within needles.

Geometry and fluid dependent models can be used with any differential pressure monitoring system. However, given the uncertainty in the physical system and changes in vessel geometry that may occur over time, it may be advantageous to use geometry independent modeling as a means of independently validating the measurements. In general, geometry independent modeling can be performed if a tractable modeling relationship can be developed, exploiting the flow dependent differential changes within the access, between the needles, as a result of changing the dialysis pump speed. The access has a blood flow rate (QA) that is dependent on numerous factors including systemic blood pressure and central venous pressure (reflecting pre- and post-access pressure gradients), access geometry (and thereby resistance), and blood viscosity to name a few. Two needles are introduced into the access lumen during conventional dialysis; one for the removal of blood (arterial) to pass through the dialysis circuit and one for the return of blood (venous) to the circulation. For the purposes of testing this differential pressure based method, the arterial needle is facing upstream and the venous needle is facing downstream. The flow through the graft or fistula remaining downstream (QD) from the arterial needle will decrease during dialysis as a function of the blood flowing through the dialysis circuit at a blood pump flow rate (QB). To the extent that the net flow through the system does not change during dialysis, this flow rate through the portion of the access between the dialysis needles during dialysis (QD) will follow the relationship QD=QA−QB. Other modeling functions can be constructed to model net changes in QA as a function of QB, but are not been considered here for the sake of simplicity.

The pressure difference between the needles will decrease as QB increases and QD decreases. While other observable signals that are predictably related to volume flow may have utility in this method, ? P (the pressure difference between the needles) was focused on. The signal ? P is measured and related mathematically to QB using a modeling function constructed for this signal F(QB) based on the measured values such that ? P=F(QB). This modeling function may take the form of any algebraic or numerical function (preferably, but not necessarily, one-to-one in the range and domain of interest): linear, polynomial, exponential or otherwise. As QD decreases with increasing QB, the signal ? P=F(QB) will decrease. As QD approaches zero, ? P will approach zero, or a known value for ? P that corresponds to zero blood flow QD. For purposes of evaluating this method, zero or near zero time-averaged mean ? P will correspond to zero volume flow QD. This value can be defined using the modeling function as the signal S0=F(0). This value for F(0) corresponds to the value for QB=QA since QD=zero. QB at the value QA can be solved by calculating the projected intercept of the modeling function where ? P=zero or the known value for ? P corresponding to zero mean flow between the needles. These calculations can be performed numerically by determining the inverse function of the modeling function or by solving them algebraically. To evaluate the method most simply, a quadratic and linear form of the relationship between ? P and access flow Q was evaluated, with two dialysis pump speeds (pump on and pump off).

For expression 1 was evaluated:

? P=C*Q, in general,

and we define:

Poff=C*QA, and

Pon=C*(QA−QB)

for Poff and Pon as the ? P for pump off and pump on. Solving for the access flow QA gives:

QA=QB/(1−Pon/Poff) (Expression 1; linear model)

For expression 2:

? P=C*(QA)̂2

and:

Poff C*(QA)̂2, and

Pon=C*(QA−QB)̂2

for Poff and Pon as the ? P for pump off and pump on. Solving for the access flow QA gives:

QA=QB/(1−v(Pon/Poff)). (Expression 2; quadratic model)

where QA depends on QB and the square root of ratio of Pon and Poff. Importantly, all of the geometric access and needle position parameters as well as the blood viscosity parameters contained in the term C have been eliminated from expression 1 and 2 above. Therefore, while these parameters may be helpful in estimating flow from pressure, a method and derived an expression have been developed for determining flow from pressure that does not depend on these factors.

### Real Time Flow Estimation

An expression for real time flow estimation (without altering the pump rate) can be tested using these experimental data. A parametric value for C (geometric and rheologic factors) can be used for C and estimated from the variable flow method.

C=Poff/QÂ2,

Substituted into Pon=C(QA−QB), and solving for QA gives:

QA=QB+v(Pon/C) (Expression 3)

where QA can be followed in real time without altering the pump rate by tracking the square root of the ratio of ? P with pump on (Pon) and C and adding this to the pump rate QB.

### An analogous relationship can be determined using expression 1, yielding

QA=QB+Pon/C (Expression 4)

should pressure vary linearly with flow. It should be noted that in practice it is anticipated that the pump may be briefly paused to re-calculate C to adjust for factors that may change during dialysis (e.g. ultrafiltration raising the hematocrit and altering viscosity) and then resuming tracking QA in real time again. Similarly, since experimental data and CFD results from a previous study demonstrated a combination of linear (laminar) and quadratic (turbulent) flow patterns, we would anticipate that a geometry independent model may represent a combination of these models. Most simply this may be an average of expression 1 and 2 to yield:

QA=(QB/2)*(1/(1−Pon/Poff)+1/(1−v(Pon/Poff)) (Expression 5; combined model)

or a more complex combination with components accounting for laminar and turbulent flow patterns. The important feature of any of these models is that they are geometry and viscosity independent. All flows are considered as time-averaged means to eliminate the need for phase information. This is an idealized model and more complex models can be derived that include additional factors.

### Results

### CFD Modeling

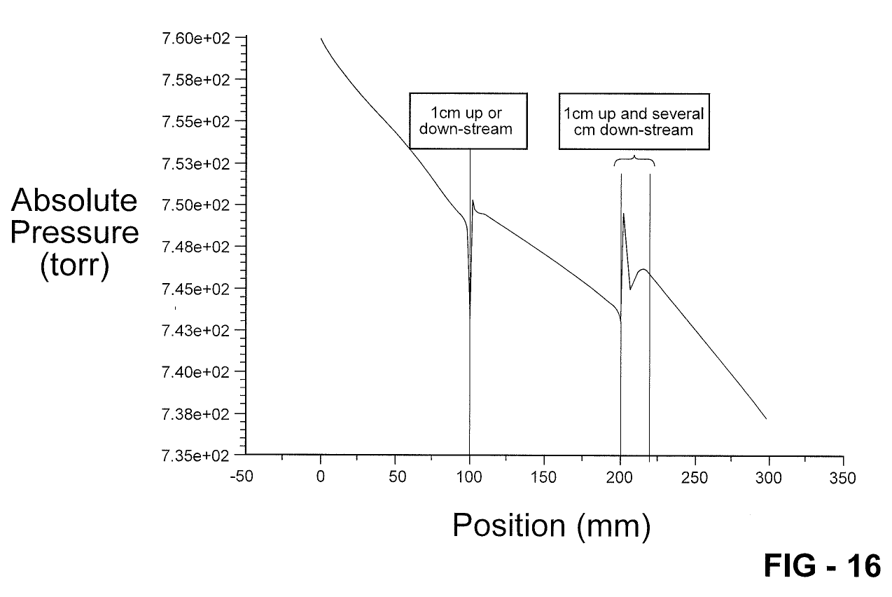

A family of curves was generated using computational fluid dynamics modeling (CFD) utilizing FLUENT software (version 6.3, Fluent, Inc, Lebanon, N.H.). The pressure at the entrance of the tubing was set at atmospheric pressure (760 mmHg). The main meshing element applied to the cylinder geometry was “Tet/Hybrid,” which specifies that the mesh is composed primarily of tetrahedral elements but may include hexahedral, pyramidal, and wedge elements where appropriate. In this model a “sink” is introduced upstream within the dialysis access to model the blood being drawn from the dialysis access through the arterial needle to the dialysis machine at a pump rate of 400 ml/min. A “source” is introduced downstream at a needle separation distance of 10 cm to model the venous needle returning blood to the dialysis access at a flow rate of 400 ml/min. Differential pressure is plotted along the y-axis with distance along the vascular access plotted along the x-axis, thereby plotting the pressure drop along the length of the access longitudinally for a family of access flows Q. The Reynold's numbers in excess of 2300 for blood exiting the dialysis needles suggests blood flow is turbulent in dialysis needles becoming laminar again within the dialysis access. Anticipated from the models derived above, FIG. 16 illustrates that the slope of ? P changes at the position of the arterial and venous needles, showing a lower slope between the needles as a function of the reduced flow in the access QD between the needles. Of importance, the CFD analysis allows estimation of regional pressure variations induced by needle tip turbulence to provide information about how close a pressure sensor may be to the needle tip and still estimate the pressure difference along the access between the needles. The flow profiles and needle tip effects were examined using CFD for access flows 400, 800, and 1200 ml/min with pump on and off at pump rates of 400 ml/min in the center of the lumen and off axis within the dialysis access conduit. CFD analysis was performed under multiple conditions, using a pressure tracing as a function of position along the inner diameter of the access and along lines parallel to the axis of the access. These showed constant features as represented in FIG. 16, demonstrating that needle tip effects were greatest within 1 cm of the needle tip upstream or downstream from the upstream facing arterial needle and within 1 cm upstream of the downstream facing venous needle, but for several centimeters downstream from the venous needle with the dialysis pump on.

Variable Flow Pressure (VFP) Modeling Results Using Flow Pressure Data

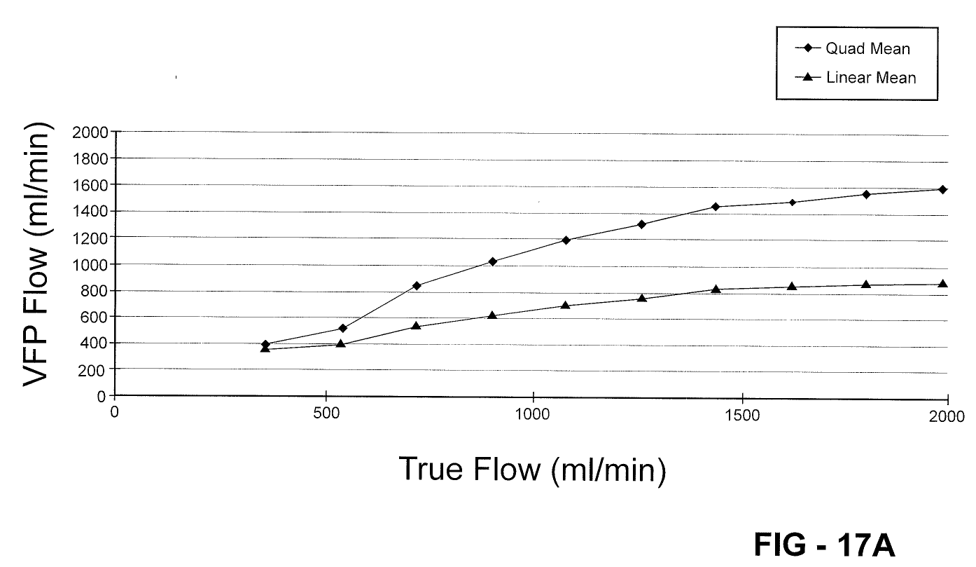

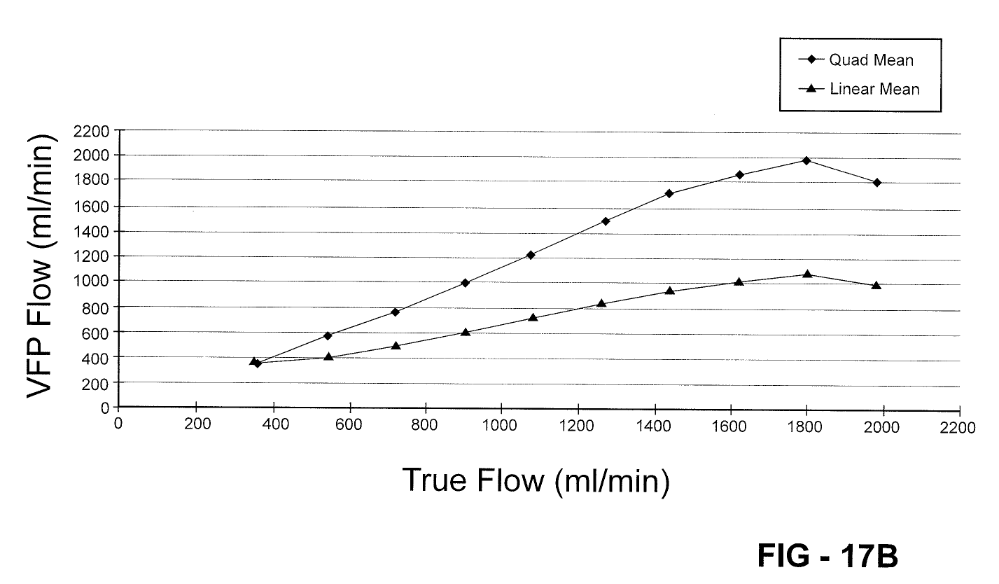

Flow pressure relationship data from a previous study were used to test the linear (laminar) and quadratic (turbulent) VFP modeling functions derived above. VFP modeling expression 1 (linear) and expression 2 (quadratic) were used to estimate flows, and results are shown in FIGS. 17a and 17b for 4.76 mm and 6.35 mm respectively, diameter access data respectively with standard deviation (10 measurements for each flow) and line of identity shown. It is important to note that these flow estimations were used using models with no geometry or viscosity dependent terms (see derivation of equations 1 and 2 above).

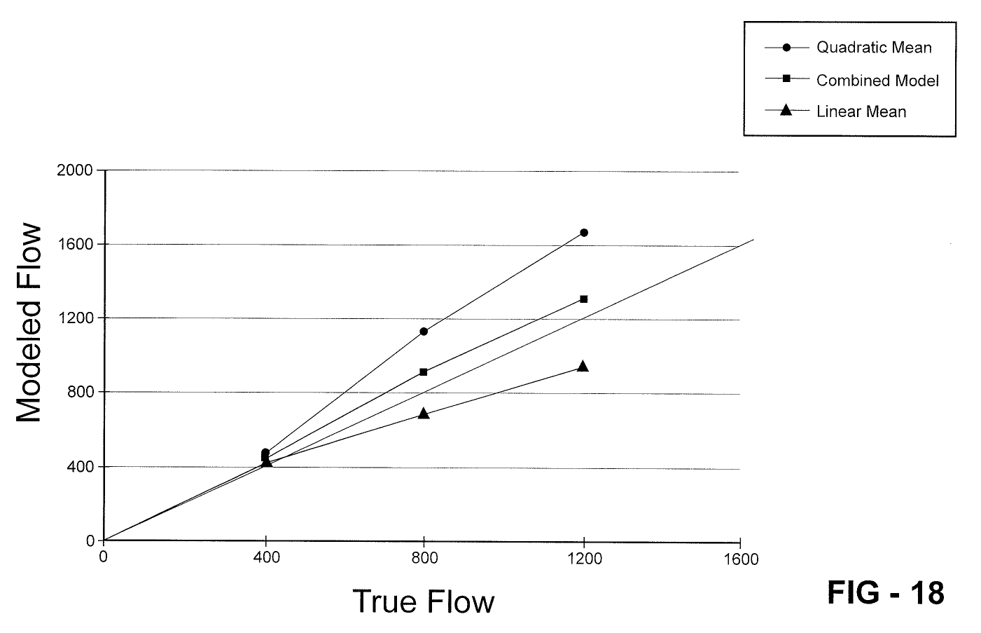

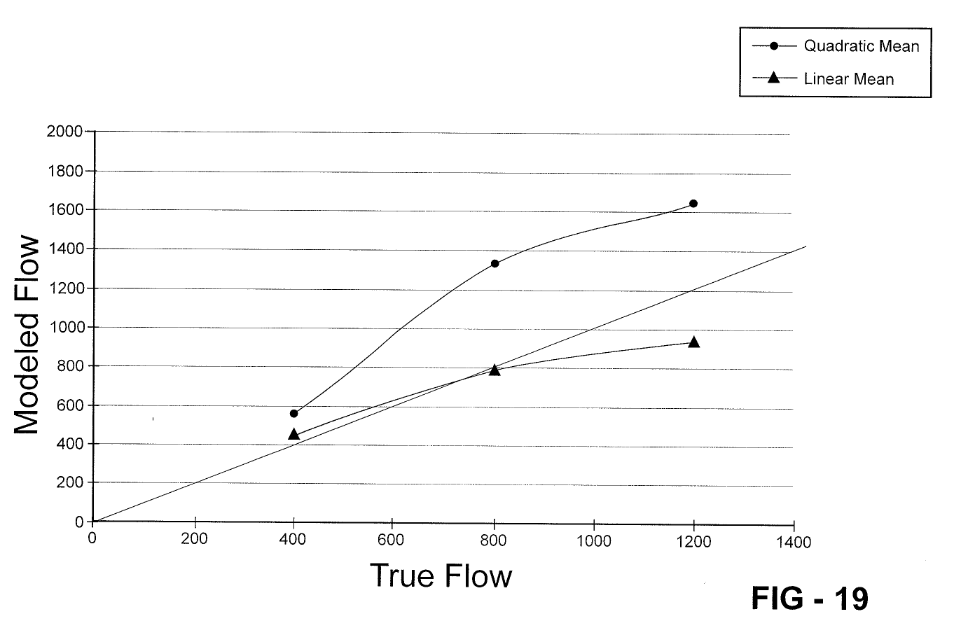

As FIG. 17 illustrates, the VFP modeling expression 1 (linear) consistently yielded lower than true volume flow results and expression 2 (quadratic model) generally yielded values equal to or above those of true flow. The VFP modeling expressions for linear (expression 1 above), quadratic (expression 2 above) and combined (expression 5 above) were tested using the experimental system in diagram 1 above with intraluminal pressure sensing. The results obtained using the experimental system described in the methods section above shows the VFP modeling results for the 4.76 diameter and 6.35 diameter access in FIGS. 18 and 19, respectively.

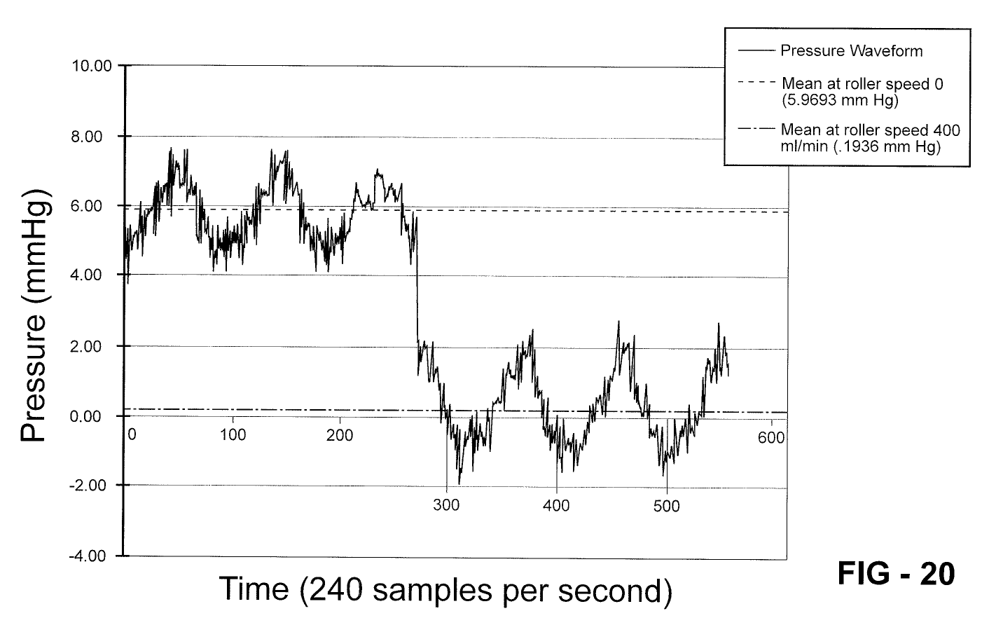

Experimental results for the VFP modeling expression 1 (linear) yielded lower than true volume flow results for the 4.76 mm diameter access and better approximated the flow in the 6.35 mm diameter access. The results for expression 2 (quadratic model) yielded values above those of true flow in both access diameters. Results were consistent for sensor needle distances 1 cm, 2 cm and 5 cm from the dialysis needle tips Results of real-time waveform information obtained during monitoring are shown in FIG. 20. The waveform information reveals that while the pump was off (pump speed zero), the pulsatility in the pressure gradient between the sensor needles corresponds to the higher pressure gradient and higher flow during systole and corresponding lower pressure gradients and flows during diastole. When the pump was turned on, an interesting phenomenon was observed; the net pressure gradient between the needles is slightly more than zero. This corresponds to slight net forward flow between the needles while the pump was on. However, what was also seen is that the systolic pressure gradient between the needles is greater than zero during systole, and the diastolic pressure gradient was less than zero. This corresponds to flow in the forward direction during systole and retrograde flow in the access during diastole. Analogous results were seen in a previous study in vivo (Weitzel W F, Rubin J M, Leavy S F, Swartz R D, et al, “Analysis of variable flow Doppler hemodialysis access flow measurements and comparison with ultrasound dilution”, Am. J. Kidney Dis., 38: 935-940, 2001) using Doppler measurements of flow between the dialysis needles during dialysis, and the pressure gradients in this experimental system corroborate the prior clinical Doppler flow findings.

The pressure gradients will correspond to alternating flow in either direction and may result in access recirculation depending on the duration of the retrograde flow and needle separation. If the retrograde distance traversed by the blood during the retrograde flow period is greater than the needle separation, then recirculation will develop. The threshold for developing recirculation can be determined by integrating the velocity of reversed (retrograde) blood flow over the time period when flow is reversed within the cardiac cycle. The velocity may be defined simplistically as v(t)=Q/A where A is the cross sectional area and Q is the flow determined from ? P. A more accurate but complicated Q can be obtained using computational fluid dynamics. For access recirculation to take place, the blood is required to traverse the distance between the needles. This distance D (v, t) for recirculation to develop can be determined by integrating:

$$D ( v , t ) = {t 2}_{t 1}^{} v(t)dt$$

Where t1 is the point in time when retrograde flow starts (when the differential pressure signal begins to become negative) during the cardiac cycle and t2 is the point in time when flow becomes forward again (when the differential pressure signal begins to become positive) during the cardiac cycle.

Obviously, many modifications and variations of the present invention are possible in light of the above teachings. It is, therefore, to be understood that within the scope of the appended claims, the invention may be practiced otherwise than as specifically described.

What is claimed:

1. A system for determining blood flow rate in a vessel which communicates blood between two locations of a patient, the system comprising:
a conduit in fluid communication with the vessel;
at least one sensor in communication with the vessel for determining differential blood pressure (? P) between two or more locations within the vessel; and
a processor operably connected to the at least one sensor for processing the ? P to obtain blood flow rate within the vessel.

2. A system according to claim 1 wherein, the conduit has a diversion point from which blood is diverted from the vessel into the conduit;

3. A system according to claim 1 further comprising a second sensor in communication with the vessel and the processor.

4. A system according to claim 1, wherein the vessel comprises a hemodialysis access or an intravascular catheter.

5. A system according to claim 1, wherein the conduit comprises an external dialysis circuit.

6. A system according to claim 1 further comprising a pump for diverting blood into the conduit.

7. A system according to claim 1, wherein the at least one sensor is externally disposed with respect to the patient.

8. A system according to claim 1, wherein the at least one sensor is disposed within the vessel.

9. A system according to claim 2, wherein the second sensor is disposed with the vessel.

10. A system according to claim 1, wherein at least one sensor is located with the conduit.

11. A system according to claim 1, wherein the at least one sensor is a miniaturized sensor.

12. A method for determining blood flow rate in a vessel which communicates blood between two locations of a patient, the method comprising:
diverting blood from the vessel at a diversion point to obtain a flow of diverted blood in a conduit;
determining differential blood pressure (? P) of the diverted blood through the conduit; and
processing the ? P to obtain blood flow rate within the vessel.

13. A method according to claim 12 further comprising the step of disposing at least one sensor in communication with the vessel for determining blood pressure change (? P) of the diverted blood through the conduit.

14. A method according to claim 12, wherein diverting blood from the vessel includes diverting blood through a hemodialysis access or an intravascular catheter.

15. A method according to claim 12 further comprising pumping the diverted blood through the conduit.

16. A method according to claim 12 further comprising returning the diverted blood to the vessel.

17. A method according to claim 12, wherein the conduit comprises an external dialysis circuit.

18. A method according to claim 12, wherein the at least one sensor is externally disposed with respect to the patient.

19. A method according to claim 12, wherein the at least one sensor is disposed within the vessel.

20. A method according to claim 12, wherein a second sensor is disposed with the vessel.

21. A method according to claim 13, wherein at least one sensor is located with the conduit.

22. A method according to claim 13, wherein at least one sensor is a miniaturized sensor.
